(19) United States
(12) Patent Application Publication
(10) Pub. No.: US 2024/0307514 A1
(43) Pub. Date: Sep. 19, 2024

(54) MODULATION OF ACMSD PROTEIN EXPRESSION

(71) Applicants: Dignity Health, San Francisco, CA (US); Board of Trustees of Michigan State University, East Lansing, MI (US); Van Andel Research Institute, Grand Rapids, MI (US)

(72) Inventors: Fredric Manfredsson, San Francisco, CA (US); Lena Brundin, San Francisco, CA (US)

(21) Appl. No.: 18/576,078

(22) PCT Filed: Jul. 1, 2022
(86) PCT No.: PCT/US2022/073392

§ 371 Date: Jan. 2, 2024

Related U.S. Application Data
(60) Provisional application No. 63/218,131, filed on Jul. 2, 2021.

(51) Int. Cl. A61K 38/51; A61K 48/00; A61P 25/28; C12N 9/88; C12N 15/86

(52) U.S. Cl. CPC A61K 38/51; A61K 48/0058; A61P 25/28; C12N 9/88; C12N 15/86; C12Y 401/01045; C12N 2750/14143

(57) ABSTRACT

Compositions for increasing the expression of ACMSD in a target cell and methods of using the compositions to provide a general protective effect in a cell or tissue in a subject, or protect a subject from or treating a disease condition associated with inflammation, oxidative stress, protein aggregation, energy failure, toxic exposure such as exposure to pollutants, or any combination thereof.

### CROSS-REFERENCE TO RELATED APPLICATIONS

This application claims priority from Provisional Application number 63/218,131, filed Jul. 2, 2021, the entire contents of which are hereby incorporated by reference.

### SEQUENCE LISTING

This application contains a Sequence Listing that has been submitted in ASCII format via EFS-Web and is hereby incorporated by reference in its entirety. The ASCII copy is named 102695-731717_2020-009.xml, and is 29.4 kilobytes in size.

### FIELD OF THE INVENTION

The present disclosure provides compositions for increasing the expression of ACMSD in a target cell and methods of using the same.

### BACKGROUND OF THE INVENTION

Numerous disease conditions are associated with pathological conditions such as abnormal inflammation, oxidative stress, protein aggregation, energy failure, and toxic exposure such as exposure to pollutants among other pathological conditions. However, the interplay of these and possibly other pathological conditions, including genetic factors, environmental conditions, immune system activity, or even pathological conditions not currently identified, is poorly understood, thereby complicating efforts to develop treatments for disease conditions. For instance, inflammation, once thought to be the consequence of the neuronal death occurring in neurodegenerative disorders such as Alzheimer's disease (AD), Parkinson's disease (PD), and amyotrophic lateral sclerosis (ALS), is now increasingly thought of as playing a participatory role in the disease process itself. Accordingly, despite the significance and potential severity of conditions that may be caused by these pathological conditions, therapeutic, prophylactic, and protective approaches are limited.

Accordingly, there is a need for treatment options capable of providing protection from disease-causing pathological conditions, and treatment for disease conditions resulting from the interplay of these conditions. In some aspects, a treatment could provide general protection against, and treatment of disease conditions that may be caused by one or more pathological conditions and the interplay of one or more of the pathological conditions.

### SUMMARY OF THE INVENTION

One aspect of the present disclosure encompasses an expression construct for expressing an aminocarboxymuconate semialdehyde decarboxylase (ACMSD) protein in a target cell. The construct comprises a promoter operably linked to a nucleic acid sequence encoding a programmable nucleic acid modification system targeted to a nucleic acid sequence in a nucleotide sequence encoding the ACMSD protein; or a promoter operably linked to a nucleotide sequence encoding the ACMSD protein. The amino acid sequence of the ACMSD protein can comprise about 75% or more, 85% or more, 95% or more, or 100% sequence identity with SEQ ID NO: 6.

The target cell can be a liver, kidney, placenta, brain, cancer cells or tumors, cells of the immune system, or any combination thereof. For instance, the target cell can be a cell of the central nervous system. The target cell can be a neural cell such as a cell of glial lineage, an astrocyte, or a glial cell.

The target cell can be in a subject having a disorder associated with inflammation, oxidative stress, protein aggregation, energy failure, toxic exposure such as exposure to pollutants, genetic factors, environmental conditions, immune system activity, or any combination thereof. The target cell can be in a subject having a neurological disorder. The neurological disorder can be Parkinson's disease, Alzheimer's disease, ALS, Huntington's disease, brain ischemia, CNS infections and autoimmune disorders, mood disorders, schizophrenia, or any combination thereof.

The promoter of the expression construct can comprise at least one tissue or cell-specific promoter control sequence. The tissue or cell-specific promoter control sequence is an interneuron specific promoter, a hippocampal promoter, a dopamine beta-hydroxylase promoter, a glutamatergic neuron promoter, a tyrosine hydroxylase promoter, a motor neuron promoter, a serotonergic promoter, a microglial promoter, an astrocyte specific promoter, an oligodendrocyte specific promoter, or any combination thereof. In some aspects, the tissue or cell-specific promoter control sequence is a microglial promoter. In other aspects, the tissue or cell-specific promoter control sequence is an astrocyte specific promoter.

In some aspects, the promoter of the expression construct comprises at least one ubiquitous promoter control sequence. The ubiquitous promoter control sequence can be a CBA/CMV promoter hybrid. The nucleic acid sequence of the expression construct can comprise about 75% or more, 85% or more, 95% or more, or 100% sequence identity with SEQ ID NO: 8, and the nucleic acid sequence of the CBA/CMV promoter hybrid can have about 75% or more, 85% or more, 95% or more, or 100% sequence identity with SEQ ID NO: 7.

In some aspects, the expression construct further comprises further comprising a nucleic acid delivery vector comprising the nucleic acid expression construct for delivering the nucleic acid expression construct to the target cell. The delivery vector can be a viral vector. The viral vector can exhibit tropism to the target cell. In some aspects, the viral vector is a recombinant adeno-associated virus (rAAV) vector encapsidating the nucleic acid construct for delivering the construct to the target cell. The rAAV vector can comprise an AAV2 capsid protein comprising a Y444F, Y500F, Y730F amino acid substitution, or any combination thereof, or corresponding substitutions in the capsid protein of another AAV serotype. The rAAV vector can also comprise an AAV2 capsid protein comprising a R585S, R588T, and R487G amino acid substitution, or any combination thereof, or corresponding substitutions in the capsid protein of another AAV serotype. In some aspects, the AAV vector comprises an expression construct expressing ACMSD, wherein the nucleic acid sequence of the AAV vector comprises about 75% or more, 85% or more, 95% or more, or 100% sequence identity with SEQ ID NO: 2.

The programmable nucleic acid modification system comprises an interfering nucleic acid molecule having a nucleotide sequence complementary to a nucleic acid sequence within a nucleic acid sequence encoding the ACMSD protein. The interfering nucleic acid molecule can be selected from an antisense molecule, siRNA molecules, single-stranded siRNA molecules, miRNA molecules, piRNA molecules, lncRNA molecules, and shRNA molecules. In some aspects, the interfering nucleic acid molecule is a shRNA, and the shRNA molecule can comprise a nucleotide sequence complementary to a target sequence within a nucleic acid sequence encoding the ACMSD protein. In some aspects, the shRNA comprises about 75% or more, 85% or more, 95% or more, or 100% sequence identity with SEQ ID NO: 15.

Another aspect of the instant disclosure encompasses an engineered vector-mediated system for expressing an aminocarboxymuconate semialdehyde decarboxylase (ACMSD) protein in a target cell. The system comprises a nucleic acid expression construct for expressing ACMSD in a target cell. The nucleic acid expression construct comprises a promoter operably linked to a nucleic acid sequence encoding a programmable nucleic acid modification system targeted to a nucleic acid sequence in a nucleotide sequence encoding the ACMSD protein; or a promoter operably linked to a nucleotide sequence encoding the ACMSD protein. The expression construct can be as described herein above.

The engineered vector-mediated system also comprises a nucleic acid delivery vector comprising the nucleic acid expression construct for delivering the nucleic acid expression construct to the target cell. The delivery system can be a viral vector. The viral vector exhibits tropism to the target cell.

The viral vector can be a recombinant adeno-associated virus (rAAV) vector encapsidating the nucleic acid construct for delivering the construct to the target cell. In some aspects, the rAAV vector comprises an AAV2 capsid protein comprising a Y444F, Y500F, Y730F amino acid substitution, or any combination thereof, or corresponding substitutions in the capsid protein of another AAV serotype. In other aspects, the rAAV vector comprises an AAV2 capsid protein comprising a R585S, R588T, and R487G amino acid substitution, or any combination thereof, or corresponding substitutions in the capsid protein of another AAV serotype. In some aspects, the engineered vector-mediated system comprises a promoter operably linked to a nucleotide sequence encoding the ACMSD protein, wherein the nucleic acid sequence of the AAV vector comprises about 75% or more, 85% or more, 95% or more, or 100% sequence identity with SEQ ID NO: 2.

An additional aspect of the instant disclosure encompasses a method of treating a disease condition associated with pathological conditions. The method comprises expressing an ACMSD protein in a target cell in a subject in need thereof by administering to the subject a therapeutically effective amount of an expression construct or a vector-mediated system for expressing an ACMSD protein in a target cell. The expression construct and the vector-mediated system can be as described herein above. The method can increase ACMSD expression or increase the expression of the ACMSD protein to levels higher than levels normally found in cells having normal or cells having defective levels of ACMSD expression.

The pathological conditions can include abnormal inflammation, oxidative stress, protein aggregation, energy failure, toxic exposure such as exposure to pollutants, genetic factors, environmental conditions, immune system activity, or any combination thereof. In some aspects, the target cell is a cell of the central nervous system. Increasing the expression of ACMSD in cells of the nervous system can provide a neurotrophic effect.

The disease condition can be associated with quinolinic acid. The disease condition can be associated with inflammation such as neuroinflammation. In some aspects, the disease condition is a neurological disorder such as Parkinson's disease, Alzheimer's disease, ALS, Huntington's disease, brain ischemia, CNS infections and autoimmune disorders, or any combination thereof. In other aspects, the disease condition is a psychiatric disorder is a mood disorder, schizophrenia, suicidality, or any combination thereof.

In some aspects, the disease condition is Huntington's disease, wherein the engineered vector-mediated system comprises a nucleic acid sequence comprising about 75% or more, 85% or more, 95% or more, or 100% sequence identity with SEQ ID NO: 2, and wherein the method comprises injecting a therapeutically effective amount of the engineered vector-mediated system in the basal ganglia of a subject in need thereof. In other aspects, the disease condition is Parkinson's disease, wherein the engineered vector-mediated system comprises a nucleic acid sequence comprising about 75% or more, 85% or more, 95% or more, or 100% sequence identity with SEQ ID NO: 2, and wherein the method comprises injecting a therapeutically effective amount of the engineered vector-mediated system in the substantia nigra of a subject in need thereof. In yet other aspects, the disease condition is Alzheimer's disease, wherein the engineered vector-mediated system comprises a nucleic acid sequence comprising about 75% or more, 85% or more, 95% or more, or 100% sequence identity with SEQ ID NO: 2, and wherein the method comprises injecting a therapeutically effective amount of the engineered vector-mediated system in the hippocampus of a subject in need thereof.

Yet another aspect of the present disclosure encompasses a method of providing a general protective or therapeutic effect in a cell or tissue in a subject in need thereof. The method comprises administering to the subject a therapeutically effective amount of an expression construct or a vector-mediated system for expressing an ACMSD protein in a target cell. The expression construct and the vector-mediated system can be as described herein above.

The method can restore a deficiency of ACMSD expression. For instance, the method can increase the expression of the ACMSD protein to levels higher than levels normally found in cells having normal or cells having defective levels of ACMSD expression.

Providing a general protective effect can comprise providing protection from pathological conditions associated with inflammation, oxidative stress, protein aggregation, energy failure, toxic exposure such as exposure to pollutants, genetic factors, environmental conditions, immune system activity, or any combination thereof. In some aspects, providing a general protective effect comprises providing protection from disease conditions associated with quinolinic acid.

In some aspects, providing a general protective effect comprises providing protection from disease conditions is associated with inflammation. The general protective effect can be a neurotrophic effect. Providing a general protective effect can comprise providing protection from disease conditions associated with neuroinflammation. Providing a general protective effect can also comprise providing protection from Parkinson's disease, Alzheimer's disease, ALS, Huntington's disease, brain ischemia, CNS infections and autoimmune disorders, mood disorders, schizophrenia, or any combination thereof.

In some aspects, the disease condition is Huntington's disease, wherein the engineered vector-mediated system comprises a nucleic acid sequence comprising about 75% or more, 85% or more, 95% or more, or 100% sequence identity with SEQ ID NO: 2, and wherein the method comprises injecting a therapeutically effective amount of the engineered vector-mediated system in the basal ganglia of a subject in need thereof. In other aspects, the disease condition is Parkinson's disease, wherein the engineered vector-mediated system comprises a nucleic acid sequence comprising about 75% or more, 85% or more, 95% or more, or 100% sequence identity with SEQ ID NO: 2, and wherein the method comprises injecting a therapeutically effective amount of the engineered vector-mediated system in the substantia nigra of a subject in need thereof. In yet other aspects, the disease condition is Alzheimer's disease, wherein the engineered vector-mediated system comprises a nucleic acid sequence comprising about 75% or more, 85% or more, 95% or more, or 100% sequence identity with SEQ ID NO: 2, and wherein the method comprises injecting a therapeutically effective amount of the engineered vector-mediated system in the hippocampus of a subject in need thereof.

One aspect of the instant disclosure encompasses a method of providing a protective or therapeutic effect against Huntington's disease in a subject in need thereof. The method comprises injecting into the basal ganglia of the subject a therapeutically effective amount of engineered vector-mediated system comprises a nucleic acid sequence comprising about 75% or more, 85% or more, 95% or more, or 100% sequence identity with SEQ ID NO: 2.

An additional aspect of the instant disclosure encompasses a method of providing a protective or therapeutic effect against Parkinson's disease in a subject in need thereof. The method comprises injecting into the substantia nigra of the subject a therapeutically effective amount of engineered vector-mediated system comprises a nucleic acid sequence comprising about 75% or more, 85% or more, 95% or more, or 100% sequence identity with SEQ ID NO: 2.

Yet another aspect of the instant disclosure encompasses a method of providing a protective or therapeutic effect against Alzheimer's disease in a subject in need thereof. The method comprises injecting into the hippocampus of the subject a therapeutically effective amount of engineered vector-mediated system comprises a nucleic acid sequence comprising about 75% or more, 85% or more, 95% or more, or 100% sequence identity with SEQ ID NO: 2.

An additional aspect of the instant disclosure encompasses a method of providing a protective or therapeutic effect against Huntington's disease in a subject in need thereof. The method comprises injecting brain wide, into the caudate/putamen (striatum) of the subject or any combination thereof a therapeutically effective amount of an engineered vector-mediated system comprising an interneuron specific promoter, a neuron specific promoter, or a glial promoter. The engineered vector-mediated system can be as described herein above.

Another aspect of the instant disclosure encompasses a method of providing a protective or therapeutic effect against Parkinson's disease in a subject in need thereof. The method comprises injecting brain wide, into the caudate/putamen, substantia nigra, cortex, brainstem, amygdala, autonomic nervous system, enteric nervous system of the subject or any combination thereof a therapeutically effective amount of an engineered vector-mediated system comprising a neuron specific promoter or a glial promoter. The engineered vector-mediated system can be as described herein above.

An additional; aspect of the instant disclosure encompasses a method of providing a protective or therapeutic effect against Alzheimer's disease in a subject in need thereof, the method comprising injecting brain wide, into the cortex, the hippocampus, the entorhinal cortex of the subject or any combination thereof a therapeutically effective amount of engineered vector-mediated system. The engineered vector-mediated system can be as described herein above.

Another aspect of the instant disclosure encompasses a method of providing a neurotrophic effect to a cell of the nervous system in a subject in need thereof. The method comprises expressing the ACMSD protein in the cell in the subject by administering to the subject a therapeutically effective amount of the expression construct or a vector-mediated system for expressing an ACMSD protein in a target cell. The expression construct and vector-mediated system can be as described herein above.

Yet another aspect of the instant disclosure encompasses a method of providing an anti-inflammatory effect to a target neuronal cell in a subject in need thereof. The method comprises expressing an ACMSD protein in the target cell in the subject by administering to the subject a therapeutically effective amount of an expression construct or a vector-mediated system for expressing an ACMSD protein in a target cell. The expression construct and vector-mediated system can be as described herein above.

One aspect of the present disclosure encompasses a library of engineered vector-mediated systems for expressing an ACMSD protein in a target cell comprising a plurality of engineered systems for expressing an ACMSD protein in a target cell. The vector-mediated systems can be as described herein above. Each of the plurality of systems can comprise a non-cell or tissue-specific delivery system and a plurality of cell-or tissue-specific ACMSD expression systems. The non-cell or tissue-specific delivery system can be a non-cell specific rAAV vector. In some aspects, the library comprises a plurality of engineered vector-mediated systems for expressing ACMSD, wherein each of the plurality of engineered vector-mediated systems comprises a plurality of cell or tissue-specific delivery systems and a protein expression system for ubiquitous expression of ACMSD. In some aspects, the cell or tissue-specific delivery system is an rAAV vector having tropism to a target cell or tissue.

An additional aspect of the present disclosure encompasses a vector-mediated system encoding an engineered system for expressing an ACMSD protein in a target cell. The engineered vector-mediated system can be as described herein above. The nucleic acid sequence of the ACMSD vector-mediated system can have about 75% or more, 85% or more, 95% or more, or 100% sequence identity with SEQ ID NO: 8.

Another aspect of the present disclosure encompasses a kit comprising one or more expression constructs and/or vector-mediated systems for expressing an ACMSD protein in a target. The expression constructs and vector-mediated systems can be as described herein above.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 Simplified diagram of the enzymes and metabolites of the kynurenine pathway. IDO, indoleamine-2,3-dioxygenase; TDO, tryptophan-2,3-dioxygenase; KATs, kynurenine aminotransferases; KMO, kynurenine-3-monooxygenase; 3-HAO, 3-hydroxyanthranilate-3,4-dioxygenase; ACMSD, aminocarboxymuconate-semialdehyde decarboxylase; QPRT, quinolinic acid phosphoribosyltransferase; NAD, nicotinamide adenine dinucleotide.

FIG. 2 depicts plots of measurements obtained during open field-testing in a Huntington's mouse model. Left to right first row: distance, horizontal activity, clockwise revolution. Left to right second row: ambulatory episode average velocity, ambulatory episode median velocity, and center distance legacy measurements are shown. WT: wild type. *Significance from WT.

FIG. 3 depicts a plot showing measured arbitrary density using one-way ANOVA data.

FIG. 4A shows mouse midbrain sections stained with tyrosine hydroxylase. WT; wild type. HET: Heterozygous. KO: Knock out

FIG. 4B is a plot quantifying tyrosine hydroxylase (TH)-containing neurons (TH-immunoreactive; TH-ir neurons) in the SNpc in WT, HET, and ACMSD knockout brains. *Significance from WT. WT; wild type. HET: Heterozygous. KO: Knock out

FIG. 4C is a plot quantifying SN-pc Nissl-stained neurons in ACMSD knockout brains. WT; wild type. HET: Heterozygous. KO: Knock out

FIG. 4D is a plot quantifying TH-ir neurons in the ventral tegmental area (VTA) in Th-ir neurons.

FIG. 4E is a plot quantifying Nissl-stained neurons in the VTA following ACMSD removal in Nissl-stained neurons.

FIG. 5A is a plot quantifying the effect of ACMSD in neuronal cells using the amphetamine (AMPH)-induced rotation test (rotational behavior following amphetamine administration in an animal model of PD). The results show improved behavior in ACMSD treated rats.

FIG. 5B is a photomicrograph of brain sections in the rat model of PD showing a qualitative assessment of TH immunoreactivity following control GFP vector delivery (panels I and II) or ACMSD vector in the SN (panel iii) and terminals (panel iv).

FIG. 5C is a plot showing stereological cell count of pilot animals.

FIG. 5D are photomicrographs of qualitative immunohistochemical assessment from animals receiving AAV-α-syn. Panel A shows staining with lba1 and Panel B shows staining with GFAP. Panels A(i) and B(i) show staining following control vector delivery. Panels A(ii) and B(ii) show staining following delivery of ACMSD vector.

FIG. 6A shows a schematic of an rAAV vector expressing ACMSD.

FIG. 6B shows a schematic of a GFP-expressing control rAAV vector derived from the vector shown in FIG. 6A, having the ACMSD DNA sequence replaced with a sequence encoding GFP.

FIG. 7A is a plot of cell count in MPTP-or PBS-treated mice receiving a GFP control vector and a vector expressing ACMSD.

FIG. 7B is a plot showing the test results using the rotarod test in mice of FIG. 7A.

FIG. 7C is a plot showing the hindlimb clasping score in mice of FIG. 7A.

FIG. 7D is a plot showing the test results using the open field test in mice of FIG. 7A.

FIG. 8 are photomicrographs showing HuC/D (pan neuronal marker) immunoreactivity in wild type animals receiving hippocampal injections with AAV-GFP (Panel A), wild type animals receiving hippocampal injections with AAV-ACMSD (Panel B), P301S animals receiving hippocampal injections with AAV-GFP (Panel C), and P301S animals receiving hippocampal injections with AAV-ACMSD (Panel D).

FIG. 9 are photomicrographs showing wild type animals receiving hippocampal injections with AAV-GFP (Panel A), wild type animals receiving hippocampal injections with AAV-ACMSD (Panel B), P301S animals receiving hippocampal injections with AAV-GFP (Panel C), and P301S animals receiving hippocampal injections with AAV-ACMSD (Panel D).

### DETAILED DESCRIPTION

The present disclosure is based in part on the surprising discovery that expression of an aminocarboxymuconate semialdehyde decarboxylase (ACMSD) can provide a general protective environment for a cell from disease-causing pathological conditions, and treatment for disease conditions resulting from these pathological conditions. Surprisingly, the inventors discovered that expression of ACMSD can support growth and development, survival, and differentiation of cells, and provide protection from, and treatment for disease conditions resulting from the interplay of these pathological conditions, including disease conditions associated with inflammation, oxidative stress, protein aggregation, energy failure, toxic exposure such as exposure to pollutants, genetic factors, environmental conditions, immune system activity, or factors not currently identified.

Importantly, the inventors surprisingly discovered that over-expression of ACMSD creates a general protective and therapeutic effect even in conditions unrelated to ACMSD or the kynurenine pathway in which ACMSD is an enzyme. Concordant with this, the inventors surprisingly discovered that over-expression of ACMSD provides this protective and therapeutic environment for cells and tissues even when wild type expression of ACMSD in a target cell is normal (not defective), or when ACMSD is expressed ectopically in the target cell. Further, over-expression of ACMSD can protect and treat conditions without there being a direct link between ACMSD or the kynerunine pathway and a specific disease condition. For instance, the inventors discovered that expression of ACMSD in the nervous system provides neurotrophic factor activity by supporting the growth, survival, and differentiation of both developing and mature neurons, thereby having a neuroprotective effect against neuroinflammation, excitotoxicity and the like.

As used herein, the terms “express” and “over-express” are used interchangeably and refer to the expression of ACMSD to a level higher than the level of expression of the protein in the cell prior to expression using the engineered systems described herein. For instance, in cells having defective levels of ACMSD, expression can restore the deficiency of ACMSD to normal levels. Expression of ACMSD can also increase the level of ACMSD protein in the cell to levels higher than levels normally found in cells having normal or defective levels of ACMSD expression. Expression or over-expression can also refer to ectopic expression of ACMSD where ACMSD is expressed at times and cells where the ACMSD is not known to have a function.

### I. Engineered System

One aspect of the present disclosure encompasses an expression construct for modifying the expression of an ACMSD protein in a target cell. The expression construct can comprise a promoter operably linked to a nucleic acid sequence encoding a programmable nucleic acid modification system targeted to a nucleic acid sequence in a nucleotide sequence encoding the ACMSD protein. Alternatively, the expression construct can comprise a promoter operably linked to a nucleotide sequence encoding the ACMSD protein. In some aspects, the engineered system can further comprise a nucleic acid delivery system to facilitate delivery of the engineered expression system to the target cell and expression of the ACMSD protein in the target cell.

### (a) ACMSD

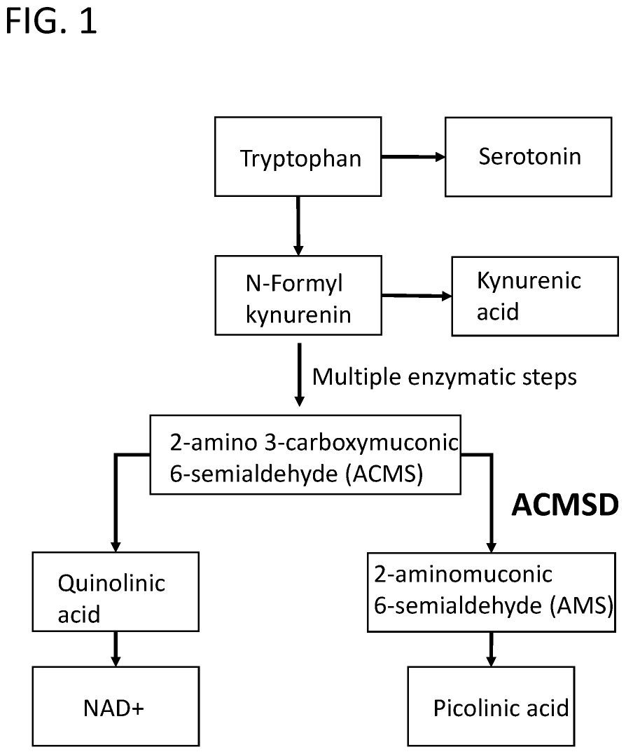

The engineered system comprises can modify the expression of ACMSD. ACMSD belongs to the amidohydrolase superfamily of proteins, whose structurally characterized members comprise a catalytically essential metal cofactor. ACMSD is an enzyme in the kynurenine pathway (FIG. 1) responsible for the catalytic breakdown of tryptophan into NAD+, generating several neuroactive metabolites in the process. These enzymes are known to be expressed in all organisms, in different tissues, including the liver, kidney, placenta, brain, cancer cells or tumors, or cells of the immune system. The ACMSD enzyme is located at a key branch-point of the pathway, where expression of ACMSD can limit the production of the toxin quinolinic acid (QA) in favor of production of picolinic acid (PIC).

QA is a potent toxin with proinflammatory and immunoregulatory properties. QA acts as a neurotoxin, excitotoxin, gliotoxin, proinflammatory mediator, pro-oxidant molecule and can alter the integrity and cohesion of the blood brain barrier. QA has been linked to fibromyalgia, stroke, traumatic brain injury, and a plethora of neurological disorders such as Parkinson's disease (PD), Alzheimer's disease (AD), Amyotrophic lateral sclerosis (ALS), Huntington's disease (HD), brain ischemia, CNS infections and autoimmune disorders, mood disorders such as suicidality and violent suicide, and schizophrenia. PIC is suspected of having neuroprotective properties, although the underlying mechanism has not been determined.

Increasing the expression of ACMSD can also decrease the levels of kynurenic acid, a metabolite in the kynurenine pathway. Kynurenic acid possesses neuroactive activity. It acts as an antiexcitotoxic and anticonvulsant, most likely through acting as an antagonist at excitatory amino acid receptors. Because of this activity, it may influence important neurophysiological and neuropathological processes. As a result, kynurenic acid has been considered for use in therapy in certain neurobiological disorders. Conversely, increased levels of kynurenic acid have also been linked to certain pathological conditions. High levels of kynurenic acid have been identified in patients suffering from tick-borne encephalitis schizophrenia and HIV-related illnesses. In all these situations increased levels were associated with confusion and psychotic symptoms. Kynurenic acid acts in the brain as a glycine-site NMDAr antagonist, key in glutamatergic neurotransmission system, which is thought to be involved in the pathophysiology and pathogenesis of schizophrenia. High levels of kynurenic acid have been identified in human urine in certain metabolic disorders, such as marked pyridoxine deficiency and deficiency/absence of kynureninase. When researchers decreased the levels of kynurenic acid in the brains of mice, their cognition was shown to improve markedly. Kynurenic acid shows neuroprotective properties. Some researchers have posited that the increased levels found in cases of neurological degradation is due to a failed attempt to protect the cells.

Accordingly, while not wishing to be bound by theory, expression of ACMSD in cells expressing enzymes of the kynurenine pathway can provide the therapeutic and protective effects by reducing the levels of QA and the toxicity associated with QA, and increasing the levels of PIC and the beneficial properties associated with PIC. Expression of ACMSD could also provide the therapeutic and protective effects of reduced levels of kynurenic acid. However, the inventors also surprisingly discovered that expression of ACMSD can provide protective and therapeutic effects from disease conditions not related to QA toxicity. For instance, the inventors discovered that over-expression of ACMSD in the central nervous system provides general anti-inflammatory effects in cells having normal levels of QA. The inventors were also surprised to discover that over-expression of ACMSD can provide these properties even in cells where ACMSD and other enzymes of the kynurenine pathway are not expressed.

An ACMSD protein of the instant disclosure can be any ACMSD protein in a kynerunine pathway of any organism, any variant thereof, or any combination thereof. Non-limiting examples of ACMSD protein variants include naturally occurring variants of an ACMSD protein, orthologs of an ACMSD protein, paralogs of an ACMSD protein, structural or functional variants of an ACMSD protein, ACMSD proteins comprising a change-of-function mutation, ACMSD proteins comprising altered expression in an organism, ACMSD proteins comprising an introduced mutation, splice variants of an ACMSD protein, truncated ACMSD proteins, or any combination thereof.

In some aspects, the ACMSD protein is a human ACMSD protein. In some aspects, the amino acid sequence of the ACMSD protein comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 6. In some aspects, the amino acid sequence of the ACMSD protein comprises about 75% or more, 85% or more, 95% or more, or 100% sequence identity with SEQ ID NO: 6.

In some aspects, the ACMSD protein is encoded by a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 5 (coding sequence). In some aspects, the ACMSD protein is encoded by a nucleotide sequence comprising about 75% or more, 85% or more, 95% or more, or 100% sequence identity with SEQ ID NO: 5.

### (b) Target Cell or Tissue

An engineered system of the instant disclosure increases the expression of ACMSD in a target cell or tissue. As explained in Section I(a) above, expression of ACMSD can provide a general protective and therapeutic effect in cells expressing normal or defective levels of the kynurenine pathway enzymes, or in cells that do not express ACMSD or any of the other enzymes of the kynurenine pathway. Accordingly, a target cell or tissue of the instant disclosure can be any cell or tissue type, or a combination of cells and tissues where a protective and therapeutic activity can result from expression of the protein. Disease conditions are described herein further below in Section II(b).

The target cell or tissue can be a cell of the nervous system such as the central nervous system and the peripheral nervous system, and organs of the body, including the reproductive organs, the sensory organs, and organs of the muscular system, digestive system, the respiratory system, the urinary system, the reproductive system, the endocrine system, the circulatory system, and the lymphatic system, among others. In some aspects, the cell is a cell of the liver, kidney, placenta, brain, cancer cells or tumors, cells of the immune system, or any combination thereof.

In some aspects, the target cell or tissue is an immune cell. For instance, the target cells can be lymphocytes, neutrophils, microglia, and monocytes/macrophages, or any combination thereof. In some aspects, the target cell or tissue is the liver, kidney, or placenta. In some aspects, the target cell or tissue type is cancer. Non-limiting examples of cancers and neoplasms can be as described below in Section IV(b).

In some aspects, the target cell is a cell of the nervous system. Non-limiting examples of cells in the nervous system include neurons, glial cells (astrocytes, oligodendrocytes, ependymal cells, satellite cells, Schwann cells, and microglia), neuroblasts, choroid plexus cells, cells related to blood vessels and coverings.

In some aspects, ACMSD is non-specifically over-expressed in any one or more cell types of the central nervous system. This can provide a neurotrophic effect in the nervous system, and generalized protection against neurological conditions not necessarily associated with a specific cell type.

### (c) Protein Expression Modification System

Any protein expression modification system capable of modifying the expression of ACMSD can be used in the instant disclosure. Non-limiting examples of suitable protein expression modification systems include expression constructs comprising nucleic acid sequences encoding an ACMSD protein, a programmable nucleic acid modification system, or a peptide, polypeptide, antibody, or antibody fragment which when expressed in the target cell or tissue type increases the level or ACMSD or the level of the enzymatic activity of the ACMSD protein.

As used herein, “protein expression” includes but is not limited to one or more of the following: transcription of a DNA sequence comprising an open reading from encoding a protein into precursor mRNA; splicing and other processing of the precursor mRNA to produce mature mRNA; mRNA stability; translation of the mature mRNA into protein (including codon usage and tRNA availability); production of a mutant protein comprising a mutation that modifies the activity of the protein, including the enzymatic activity; and glycosylation and/or other modifications of the translation product, if required for proper expression and function.

In some aspects, the protein expression system is a protein expression construct expressing ACMSD. Expression constructs can be as described in Section II. In some aspects, the ACMSD protein expression modification system is a programmable nucleic acid modification system targeted to a sequence within a nucleic acid sequence encoding the ACMSD protein as well as any nucleic acid sequence that contains information for the regulated biosynthesis of an RNA product, a protein, or both including promoters, exons, introns, and other untranslated regions that control expression. As used herein, a “programmable nucleic acid modification system” is a system capable of targeting and modifying the nucleic acid or modifying the expression or stability of a nucleic acid to alter a protein or the expression of a protein encoded by the nucleic acid. The programmable nucleic acid modification system can be an interfering nucleic acid molecule, or a nucleic acid editing system.

In some aspects, the programmable expression modification system comprises an interfering nucleic acid (RNAi) molecule having a nucleotide sequence complementary to a target sequence within a gene encoding the polypeptide or polynucleotide used to inhibit expression of the polypeptide or polynucleotide. RNAi molecules generally act by forming a heteroduplex with a target RNA molecule, which is selectively degraded or “knocked down,” hence inactivating the target RNA. Under some conditions, an interfering RNA molecule can also inactivate a target transcript by repressing transcript translation and/or inhibiting transcription. An interfering RNA is more generally said to be “targeted against” a biologically relevant target, such as a protein, when it is targeted against the nucleic acid encoding the target. For example, an interfering RNA molecule has a nucleotide (nt) sequence which is complementary to an endogenous mRNA of a target gene sequence. Thus, given a target gene sequence, an interfering RNA molecule can be prepared which has a nucleotide sequence at least a portion of which is complementary to a target gene sequence. When introduced into cells, the interfering RNA binds to the target mRNA, thereby functionally inactivating the target mRNA and/or leading to degradation of the target mRNA.

Interfering RNA molecules include, inter alia, small interfering RNA (siRNA), microRNA (miRNA), piwi-interacting RNA (piRNA), long non-coding RNAs (long ncRNAs or lncRNAs), and small hairpin RNAs (shRNA). lncRNAs are widely expressed and have key roles in gene regulation. Depending on their localization and their specific interactions with DNA, RNA and proteins, lncRNAs can modulate chromatin function, regulate the assembly and function of membraneless nuclear bodies, alter the stability and translation of cytoplasmic mRNAs, and interfere with signaling pathways. Piwi-interacting RNA (piRNA) is the largest class of small non-coding RNA molecules expressed in animal cells. piRNAs regulate gene expression through interactions with piwi-subfamily Argonaute proteins. SiRNA are double-stranded RNA molecules, preferably about 19-25 nucleotides in length. When transfected into cells, siRNA inhibit the target mRNA transiently until they are also degraded within the cell. MiRNA and siRNA are biochemically and functionally indistinguishable. Both are about the same in nucleotide length with 5′-phosphate and 3′-hydroxyl ends, and assemble into an RNA-induced silencing complex (RISC) to silence specific gene expression. siRNA and miRNA are distinguished based on origin. siRNA is obtained from long double-stranded RNA (dsRNA), while miRNA is derived from the double-stranded region of a 60-70 nt RNA hairpin precursor. Small hairpin RNAs (shRNA) are sequences of RNA, typically about 50-80 base pairs, or about 50, 55, 60, 65, 70, 75, or about 80 base pairs in length, that include a region of internal hybridization forming a stem loop structure consisting of a base-pair region of about 19-29 base pairs of double-strand RNA (the stem) bridged by a region of single-strand RNA (the loop) and a short 3′ overhang. shRNA molecules are processed within the cell to form siRNA which in turn knock down target gene expression. shRNA can be incorporated into plasmid vectors and integrated into genomic DNA for longer-term or stable expression, and thus longer knockdown of the target mRNA.

Interfering nucleic acid molecules can contain RNA bases, non-RNA bases, or a mixture of RNA bases and non-RNA bases. For example, interfering nucleic acid molecules provided herein can be primarily composed of RNA bases but also contain DNA bases or non-naturally occurring nucleotides. The interfering nucleic acids can employ a variety of oligonucleotide chemistries. Examples of oligonucleotide chemistries include, without limitation, peptide nucleic acid (PNA), linked nucleic acid (LNA), phosphorothioate, 2′O-Me-modified oligonucleotides, and morpholino chemistries, including combinations of any of the foregoing. In general, PNA and LNA chemistries can utilize shorter targeting sequences because of their relatively high target binding strength relative to 2′O-Me oligonucleotides. Phosphorothioate and 2′O-Me-modified chemistries are often combined to generate 2′O-Me-modified oligonucleotides having a phosphorothioate backbone.

In some aspects, the protein expression modification system is a shRNA molecule comprising a nucleotide sequence complementary to a target sequence within a nucleic acid sequence encoding an ACMSD protein. In some aspects, the nucleic acid sequence of the shRNA comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 15. In some aspects, the nucleic acid sequence of the shRNA comprises about 75% or more, 85% or more, 95% or more, or 100% sequence identity with SEQ ID NO: 15. In some aspects, the nucleic acid sequence of the shRNA comprises a nucleic acid sequence of SEQ ID NO: 15.

In some aspects, the programmable nucleic acid modification system is a nucleic acid editing system. Such modification systems can be used to edit DNA or RNA sequences to repress transcription or translation of an mRNA, and/or produce mutant proteins with reduced or increased activity or stability. Nucleic acid editing systems generally comprise a programmable, sequence-specific nucleic acid-binding domain and an active or inactivated nuclease domain. Non-limiting examples of programmable nucleic acid modification systems include, without limit, an RNA-guided clustered regularly interspersed short palindromic repeats (CRISPR)/CRISPR-associated (Cas) (CRISPR/Cas) nuclease system, a CRISPR/Cpf1 nuclease system, a zinc finger nuclease (ZFN), a transcription activator-like effector nuclease (TALEN), a meganuclease, a ribozyme, or a programmable DNA binding domain linked to a nuclease domain. Other suitable programmable nucleic acid modification systems will be recognized by individuals skilled in the art. Non-limiting examples of editing systems can be as described in Section I(c)i-v herein below.

The programmable nucleic acid-binding domain may be designed or engineered to recognize and bind different nucleic acid sequences. In some aspects, the nucleic acid-binding domain is mediated by interaction between a protein and the target nucleic acid sequence. Thus, the nucleic acid-binding domain may be programmed to bind a nucleic acid sequence of interest by protein engineering. In other editing systems, the nucleic acid-binding domain is mediated by a guide nucleic acid that interacts with a protein of the targeting nuclease and the target nucleic acid sequence. In such instances, the programmable nucleic acid-binding domain may be targeted to a nucleic acid sequence of interest by designing the appropriate guide nucleic acid. When the editing system comprises more than one component, such as a protein and a guide nucleic acid, the multi-component editing system can be modular, in that the different components may optionally be distributed among two or more nucleic acid constructs as described herein.

i. CRISPR Nuclease Systems.

The programmable nucleic acid modification system can be an RNA-guided CRISPR nuclease system. The CRISPR system is guided by a guide RNA to a target sequence at which a protein of the system introduces a double-stranded break in a target nucleic acid sequence.

The CRISPR nuclease system may be derived from any type of CRISPR nuclease system, including a type I (i.e., IA, IB, IC, ID, IE, or IF), type II (i.e., IIA, IIB, or IIC), type III (i.e., IIIA or IIIB), or type V CRISPR system. The CRISPR/Cas system may be from Streptococcus sp. (e.g., Streptococcus pyogenes), Campylobacter sp. (e.g., Campylobacter jejuni), Francisella sp. (e.g., Francisella novicida), Acaryochloris sp., Acetohalobium sp., Acidaminococcus sp., Acidithiobacillus sp., Alicyclobacillus sp., Allochromatium sp., Ammonifex sp., Anabaena sp., Arthrospira sp., Bacillus sp., Burkholderiales sp., Caldicelulosiruptor sp., Candidatus sp., Clostridium sp., Crocosphaera sp., Cyanothece sp., Exiguobacterium sp., Finegoldia sp., Ktedonobactersp., Lactobacillus sp., Lyngbya sp., Marinobacter sp., Methanohalobium sp., Microscillasp., Microcoleus sp., Microcystis sp., Natranaerobius sp., Neisseria sp., Nitrosococcussp., Nocardiopsis sp., Nodularia sp., Nostoc sp., Oscillatoria sp., Polaromonas sp., Pelotomaculum sp., Pseudoalteromonas sp., Petrotoga sp., Prevotella sp., Staphylococcus sp., Streptomyces sp., Streptosporangium sp., Synechococcus sp., or Thermosipho sp.

Non-limiting examples of suitable CRISPR systems include CRISPR/Cas systems, CRISPR/Cpf systems, CRISPR/Cmr systems, CRISPR/Csa systems, CRISPR/Csb systems, CRISPR/Csc systems, CRISPR/Cse systems, CRISPR/Csf systems, CRISPR/Csm systems, CRISPR/Csn systems, CRISPR/Csx systems, CRISPR/Csy systems, CRISPR/Csz systems, and derivatives or variants thereof. The CRISPR system may be a type II Cas9 protein, a type V Cpf1 protein, or a derivative thereof. In some aspects, an endonuclease is a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a homolog thereof, a recombination of the naturally occurring molecule thereof, a codon-optimized version thereof, or a modified version thereof, or any combination thereof. In some embodiments, an endonuclease may introduce one or more single-stranded breaks (SSBs) and/or one or more double-stranded breaks (DSBs).

In general, a protein of the CRISPR system comprises a RNA recognition and/or RNA binding domain, which interacts with the guide RNA. A protein of the CRISPR system also comprises at least one nuclease domain having endonuclease activity. For example, a Cas9 protein may comprise a RuvC-like nuclease domain and an HNH-like nuclease domain, and a Cpf1 protein may comprise a RuvC-like domain. A protein of the CRISPR system may also comprise DNA binding domains, helicase domains, RNase domains, protein-protein interaction domains, dimerization domains, as well as other domains.

A protein of the CRISPR system may be associated with guide RNAs (gRNA). The guide RNA may be a single guide RNA (i.e., sgRNA), or may comprise two RNA molecules (i.e., crRNA and tracrRNA). The guide RNA interacts with a protein of the CRISPR system to guide it to a target site in the DNA. The target site has no sequence limitation except that the sequence is bordered by a protospacer adjacent motif (PAM). For example, PAM sequences for Cas9 include 3′-NGG, 3′-NGGNG, 3′-NNAGAAW, and 3′-ACAY, and PAM sequences for Cpf1 include 5′-TTN (wherein N is defined as any nucleotide, W is defined as either A or T, and Y is defined as either C or T). Each gRNA comprises a sequence that is complementary to the target sequence (e.g., a Cas9 gRNA may comprise GN17-20GG). The gRNA may also comprise a scaffold sequence that forms a stem loop structure and a single-stranded region. The scaffold region may be the same in every gRNA. In some aspects, the gRNA may be a single molecule (i.e., sgRNA). In other aspects, the gRNA may be two separate molecules. Those skilled in the art are familiar with gRNA design and construction, e.g., gRNA design tools are available on the internet or from commercial sources.

A CRISPR system may comprise one or more nucleic acid binding domains associated with one or more, or two or more selected guide RNAs used to direct the CRISPR system to one or more, or two or more selected target nucleic acid loci. For instance, a nucleic acid binding domain may be associated with one or more, or two or more selected guide RNAs, each selected guide RNA, when complexed with a nucleic acid binding domain, causing the CRISPR system to localize to the target of the guide RNA.

ii. CRISPR Nickase Systems.

The programmable nucleic acid modification system can also be a CRISPR nickase system. CRISPR nickase systems are similar to the CRISPR nuclease systems described above except that a CRISPR nuclease of the system is modified to cleave only one strand of a double-stranded nucleic acid sequence. Thus, a CRISPR nickase, in combination with a guide RNA of the system, may create a single-stranded break or nick in the target nucleic acid sequence. Alternatively, a CRISPR nickase in combination with a pair of offset gRNAs may create a double-stranded break in the nucleic acid sequence.

A CRISPR nuclease of the system may be converted to a nickase by one or more mutations and/or deletions. For example, a Cas9 nickase may comprise one or more mutations in one of the nuclease domains, wherein the one or more mutations may be D10A, E762A, and/or D986A in the RuvC-like domain, or the one or more mutations may be H840A (or H839A), N854A and/or N863A in the HNH-like domain.

iii. Catalytically Dead CRISPR Endonuclease Systems.

The programmable nucleic acid modification system can also be a catalytically dead CRISPR endonuclease system. Catalytically dead CRISPR systems are similar to the CRISPR nuclease systems described above except that a nuclease of the CRISPR of the system is modified to inactivate its endonuclease activity. Thus, a catalytically dead CRISPR system, in combination with a guide RNA, can target specific nucleic acid sequences complementary to the gRNA with PAM sequences that allow the catalytically dead nuclease to bind the target nucleic acid sequences.

may create a single-stranded break or nick in the target nucleic acid sequence. Alternatively, a CRISPR nickase in combination with a pair of offset gRNAs may create offset double-stranded break in the nucleic acid sequence. For example, Cas9 ordinarily has 2 endonuclease domains called the RuvC and HNH domains. The point mutations D10A and H840A change 2 important residues for endonuclease activity that ultimately results in its deactivation. Although dCas9 lacks endonuclease activity, it is still capable of binding to its guide RNA and the DNA strand that is being targeted without inducing double-strand breaks. Fusion of diverse effector domains to dCas proteins empowers the CRISPR/dCas system as a multifunctional platform for regulation nucleic acid sequences and proteins, epigenetic regulation and sequence-specific imaging.

iv. ssDNA-Guided Argonaute Systems.

Alternatively, the programmable nucleic acid modification system may comprise a single-stranded DNA-guided Argonaute endonuclease. Argonautes (Agos) are a family of endonucleases that use 5′-phosphorylated short single-stranded nucleic acids as guides to cleave nucleic acid targets. Some prokaryotic Agos use single-stranded guide DNAs and create double-stranded breaks in nucleic acid sequences. The ssDNA-guided Ago endonuclease may be associated with a single-stranded guide DNA.

The Ago endonuclease may be derived from Alistipes sp., Aquifex sp., Archaeoglobus sp., Bacteriodes sp., Bradyrhizobium sp., Burkholderia sp., Cellvibriosp., Chlorobium sp., Geobacter sp., Mariprofundus sp., Natronobacterium sp., Parabacteriodes sp., Parvularcula sp., Planctomyces sp., Pseudomonas sp., Pyrococcus sp., Thermus sp., or Xanthomonas sp. For instance, the Ago endonuclease may be Natronobacterium gregoryi Ago (NgAgo). Alternatively, the Ago endonuclease may be Thermus thermophilus Ago (TtAgo). The Ago endonuclease may also be Pyrococcus furiosus (PfAgo).

The single-stranded guide DNA (gDNA) of an ssDNA-guided Argonaute system is complementary to the target site in the nucleic acid sequence. The target site has no sequence limitations and does not require a PAM. The gDNA generally ranges in length from about 15-30 nucleotides. The gDNA may comprise a 5′ phosphate group. Those skilled in the art are familiar with ssDNA oligonucleotide design and construction.

v. Zinc Finger Nucleases.

The programmable nucleic acid modification system can be a zinc finger nuclease (ZFN). A ZFN comprises a DNA-binding zinc finger region and a nuclease domain. The zinc finger region may comprise from about two to seven zinc fingers, for example, about four to six zinc fingers, wherein each zinc finger binds three nucleotides. The zinc finger region may be engineered to recognize and bind to any DNA sequence. Zinc finger design tools or algorithms are available on the internet or from commercial sources. The zinc fingers may be linked together using suitable linker sequences.

A ZFN also comprises a nuclease domain, which may be obtained from any endonuclease or exonuclease. Non-limiting examples of endonucleases from which a nuclease domain may be derived include, but are not limited to, restriction endonucleases and homing endonucleases. The nuclease domain may be derived from a type II-S restriction endonuclease. Type II-S endonucleases cleave DNA at sites that are typically several base pairs away from the recognition/binding site and, as such, have separable binding and cleavage domains. These enzymes generally are monomers that transiently associate to form dimers to cleave each strand of DNA at staggered locations. Non-limiting examples of suitable type II-S endonucleases include BfiI, BpmI, BsaI, BsgI, BsmBI, BsmI, BspMI, FokI, MbolI, and SapI. The type II-S nuclease domain may be modified to facilitate dimerization of two different nuclease domains. For example, the cleavage domain of FokI may be modified by mutating certain amino acid residues. By way of non-limiting example, amino acid residues at positions 446, 447, 479, 483, 484, 486, 487, 490, 491, 496, 498, 499, 500, 531, 534, 537, and 538 of FokI nuclease domains are targets for modification. For example, one modified FokI domain may comprise Q486E, 1499L, and/or N496D mutations, and the other modified FokI domain may comprise E490K, 1538K, and/or H537R mutations.

vi. Transcription Activator-Like Effector Nuclease Systems.

The programmable nucleic acid modification system can also be a transcription activator-like effector nuclease (TALEN) or the like. TALENs comprise a DNA-binding domain composed of highly conserved repeats derived from transcription activator-like effectors (TALEs) that are linked to a nuclease domain. TALEs are proteins secreted by plant pathogen Xanthomonas to alter transcription of DNA sequences in host plant cells. TALE repeat arrays may be engineered via modular protein design to target any DNA sequence of interest. Other transcription activator-like effector nuclease systems may comprise, but are not limited to, the repetitive sequence, transcription activator like effector (RipTAL) system from the bacterial plant pathogenic Ralstonia solanacearum species complex (Rssc). The nuclease domain of TALEs may be any nuclease domain as described above in Section (I)(d)(i).

vii. Meganucleases or Rare-Cutting Endonuclease Systems.

The programmable nucleic acid modification system can be a meganuclease or derivative thereof. Meganucleases are endodeoxyribonucleases characterized by long recognition sequences, i.e., the recognition sequence generally ranges from about 12 base pairs to about 45 base pairs. As a consequence of this requirement, the recognition sequence generally occurs only once in any given genome. Among meganucleases, the family of homing endonucleases named LAGLIDADG has become a valuable tool for the study of genomes and genome engineering. Non-limiting examples of meganucleases that may be suitable for the instant disclosure include I-SceI, I-CreI, I-DmoI, any variant thereof, or any combination thereof. A meganuclease may be targeted to a specific nucleic acid sequence by modifying its recognition sequence using techniques well known to those skilled in the art.

The programmable targeting nuclease can be a rare-cutting endonuclease or derivative thereof. Rare-cutting endonucleases are site-specific endonucleases whose recognition sequence occurs rarely in a genome, such as only once in a genome. The rare-cutting endonuclease may recognize a 7-nucleotide sequence, an 8-nucleotide sequence, or longer recognition sequence. Non-limiting examples of rare-cutting endonucleases include NotI, AscI, PacI, AsiSI, SbfI, and FseI.

viii. Optional Additional Domains.

The programmable nucleic acid modification system may further comprise at least one nuclear localization signal (NLS), at least one cell-penetrating domain, at least one reporter domain, and/or at least one linker.

In general, an NLS comprises a stretch of basic amino acids. Nuclear localization signals are known in the art (see, e.g., Lange et al., J. Biol. Chem., 2007, 282:5101-5105). The NLS may be located at the N-terminus, the C-terminal, or in an internal location of the fusion protein.

A cell-penetrating domain may be a cell-penetrating peptide sequence derived from the HIV-1 TAT protein. The cell-penetrating domain may be located at the N-terminus, the C-terminal, or in an internal location of the fusion protein.

A programmable nucleic acid modification system can further comprise at least one linker. For example, the binding domain, the nuclease domain, and other optional domains can be linked via one or more linkers. The linker can be flexible (e.g., comprising small, non-polar (e.g., Gly) or polar (e.g., Ser, Thr) amino acids). Examples of suitable linkers are well known in the art, and programs to design linkers are readily available (Crasto et al., Protein Eng., 2000, 13(5):3096-312). In alternate aspects, the programmable targeting nuclease, the cell cycle regulated protein, and other optional domains may be linked directly.

A programmable nucleic acid modification system can further comprise an organelle localization or targeting signal that directs a molecule to a specific organelle. A signal may be polynucleotide or polypeptide signal, or may be an organic or inorganic compound sufficient to direct an attached molecule to a desired organelle. Organelle localization signals can be as described in U.S. Patent Publication No. 20070196334, the disclosure of which is incorporated herein in its entirety.

In some aspects, the programmable nucleic acid modification system is a CRISPR/Cas tool modified for transcriptional regulation of a locus. In some aspects, the programmable nucleic acid modification system is a CRISPR/Cas transcriptional regulator driven by cell-specific promoters using a catalytically dead effector (dCAS9) to modulate transcription of nucleic acid sequences encoding an ACMSD protein.

### (d) Delivery Systems

In some aspects, the engineered system for modifying the expression of an ACMSD protein further comprises a nucleic acid delivery system comprising the nucleic acid construct for delivering the nucleic acid construct to the target cell. A nucleic acid delivery system can be any system capable of delivering the nucleic acid construct to the target cell or tissue. Non-limiting examples of delivery systems include viral and non-viral constructs, and/or vectors to introduce the programmable nucleic acid modification system into a cell or organism. In some aspects, the delivery system has tropism to the target cell or tissue.

In some aspects, the nucleic acid delivery system is a non-viral vector. Non-viral vectors can include plasmids, linear DNA fragments, transposons, and artificial chromosomes and the like, that may or may not be able to replicate autonomously or integrate into a chromosome of a host cell. Delivery/administration of non-viral vectors can be as described in Section IV(a).

In some aspects, the nucleic acid delivery system is a viral vector. The viral vector can be a bacteriophage, pro-viruses, phagemids, an adenovirus vector; an adeno-associated virus (AAV) vector; a pox virus vector, such as a fowlpox virus vector; an alpha virus vector; a baculoviral vector; a herpes virus vector; a retrovirus vector, such as a lentivirus vector; a Modified Vaccinia virus Ankara vector; a Ross River virus vector; a Sindbis virus vector; a Semliki Forest virus vector; and a Venezuelan Equine Encephalitis virus vector.

In some aspects, the vector is a lentiviral vector. A recombinant lentiviral vector is capable of transducing a target cell with a nucleotide of interest. Once within the cell, the RNA genome from the vector particle is reverse transcribed into DNA and integrated into the genome of the target cell. The lentiviral vector can be derived from or may be derivable from any suitable lentivirus. The lentiviral vector can be derived from primate or non-primate lentiviruses. Examples of primate lentiviruses include but are not limited to the human immunodeficiency virus (HIV) and the simian immunodeficiency virus (SrV). The non-primate lentiviral group includes the prototype “slow virus” visna/maedi virus (VMV), as well as the related caprine arthritis-encephalitis virus (CAEV), equine infectious anemia virus (EIAV), feline immunodeficiency virus (FIV), and bovine immunodeficiency virus (BIV).

In other aspects, the viral vector is a herpes simplex virus (HSV) vector. The genome of the type-1 (HSV-1) is about 150 kb of linear, double-stranded DNA, featuring about 70 genes. Many viral genes can be deleted without the virus losing its ability to propagate. The “immediately early” (IE) genes are transcribed first. They encode trans-acting factors which regulate expression of other viral genes. The “early” (E) gene products participate in replication of viral DNA. The late genes encode the structural components of the virion as well as proteins, which turns on transcription of the IE and E genes, or disrupt host cell protein translation.

In yet other aspects, the delivery system is an adeno-associated virus (AAV) vector encapsidating the nucleic acid construct for delivering the construct to the target cell or tissue type. The adenovirus genome consists of about 36 kb of double-stranded DNA. Adenoviruses target airway epithelial cells, but are also capable of infecting neurons. Recombinant adenovirus vectors have been used as gene transfer vehicles for non-dividing cells. These vectors are similar to recombinant HSV vectors, since the adenovirus E1a immediate-early gene is removed but most viral genes are retained. Since the E1a gene is small (roughly 1.5 kb) and the adenovirus genome is approximately one-third of the size of the HSV genome, other non-essential adenovirus genes are removed in order to insert a foreign gene within the adenovirus genome.

Briefly, AAV vectors generally comprise the AAV inverted terminal repeats (ITRs) of the virus flanking heterologous nucleic acid sequences of interest. AAV ITRs contain all cis-acting elements involved in AAV genome rescue, replication, and packaging. Accordingly, the ITRs can be segregated from the viral encoding regions allowing for AAV vector design that comprises only the ITRs of the virus flanking heterologous nucleic acid sequences of interest. Generally, rAAV particles are generated by transfecting producer cells with a plasmid (AAV cis-plasmid) containing a cloned AAV vector, and a separate construct expressing in trans the viral rep and cap genes. The adenovirus helper factors, such as E1A, E1B, E2A, E4ORF6 and VA RNAs, can be provided by either adenovirus infection or transfecting into production cells a third plasmid that provides these adenovirus helper factors.

AAV vectors can be constructed using known techniques to provide at least the operatively linked components of control elements including a transcriptional initiation region, an exogenous nucleic acid molecule, a transcriptional termination region, and at least one post-transcriptional regulatory sequence. The control elements are selected to be functional in the targeted cell. and/or in combination with incorporation of mutations that enhance specific infectivity.

In some aspects, the delivery system has tropism to and provides transduction in a desired target cell or tissue type. In some aspects, the target cell or tissue type is a cell of the central nervous system. The use of AAV vectors to deliver nucleic acids into the brain is well known in the art. (See, e.g., U.S. Pat. No. 8,487,088, which is incorporated by reference herein in its entirety). The AAV can be any AAV serotype, including a serotype selected from AAV1, AAV2, AAV3, AAV4, AAV5, AAV6, AAV7, AAV8, AAVrh family (e.g., AAVrh8, AAVrh10, AAVrh39, or AAVrh43), or AAVanc family. Included are rAAV vectors comprising an AAV2 capsid comprising mutations with improved transduction efficiency of a desired target cell type. Non-limiting examples of capsid mutations having improved transduction efficiency include the Y444F, Y500F, Y730F, T491V, R585S, R588T, R487G amino acid substitutions, or corresponding substitutions in the capsid protein of another AAV serotype, in various combinations and/or in combination with incorporation of mutations that enhance tropism of the virus to a desired target cell or tissue type, and methods of generating the library. Such mutations can include insertion of one or more peptides for targeting the virus to a cell or tissue type. rAAV vectors suitable for the instant disclosure can be as described in International Patent Application No. PCT/US2021/023314, the entire disclosure of which is incorporated herein by reference. When the disorder is a neurological or psychiatric disorder, the AAV capsid can be AAV-PHP.eB that can provide brain-wide transduction.

In some aspects, the rAAV comprises an AAV2 capsid comprising mutations inhibiting the canonical HSPG binding site such as the R585S, R588T, and R487G amino acid substitutions in various combinations, or corresponding substitutions in the capsid protein of another AAV serotype. In some aspects, the rAAV comprises AAV capsids comprising the mutations inhibiting the canonical HSPG binding site such as the Y444F, Y500F, Y730F amino acid substitutions in various combinations, or analogous substitutions in the capsid protein of another AAV serotype.

In some aspects, the delivery system is an AAV vector having tropism or improved efficiency in targeting a neuronal cell, a glial cell or an astrocyte. Glial cells can be a cell of glial lineage or an activated glia. In other aspects, the delivery system is an AAV vector having tropism to activated astrocytes.

In some aspects, the delivery system is an AAV vector comprising AAV ITRs flanking an expression construct expressing ACMSD. The expression construct expressing ACMSD can be as described in Section II herein below. In some aspects, the AAV vector comprises an expression construct expressing ACMSD, wherein the nucleic acid sequence of the AAV vector comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 2. In some aspects, the AAV vector comprises an expression construct expressing ACMSD, wherein the nucleic acid sequence of the AAV vector comprises about 75% or more, 85% or more, 95% or more, or 100% sequence identity with SEQ ID NO: 2.

In some aspects, the nucleic acid sequence of the AAV ITRs comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 3. In some aspects, the nucleic acid sequence of the AAV ITRs comprises about 75% or more, 85% or more, 95% or more, or 100% sequence identity with SEQ ID NO: 3.

### II. Nucleic Acid Constructs

Protein expression modification systems of the instant disclosure can be encoded by a nucleic acid construct. The expression construct generally comprises nucleic acid coding sequences operably linked to at least one promoter control sequence for expression of the protein modification system in a target cell or tissue. Promotor control sequences can include constitutive, ubiquitous, regulated, cell-or tissue-specific promoters, or any combination thereof.

Suitable eukaryotic constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or combinations of any of the foregoing. Examples of suitable eukaryotic regulated promoter control sequences include, without limit, those regulated by heat shock, metals, steroids, antibiotics, or alcohol. Non-limiting examples of tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, NphsI promoter, OG-2 promoter, SP-B promoter, SYN1 promoter, and WASP promoter. Promoter control sequences can also be promoter control sequences of the gene of interest, such that the expression pattern of the one or more nucleic acid constructs matches the expression pattern of the gene of interest. The promoter sequence can be wild type or it can be modified for more efficient or efficacious expression. In some aspects, the promoter is CD38, F4/80, GFAP for glial subtypes, CMV and truncated CAG ubiquitous promoter, or any combination thereof. Non-limiting examples of microglial-specific promoters include CD38, cd11b, cd68, and F4/80. Non-limiting examples of astrocyte-specific promoters include GFAP or gfaABC1D, and gfa2. Non-limiting examples of oligodendrocyte-and schwann cell-specific promoters include myelin basic protein promoter and myelin-associated glycoprotein promoter.

The nucleic acid constructs can comprise additional expression control sequences (e.g., enhancer sequences, Kozak sequences, polyadenylation sequences, transcriptional termination sequences, etc.), selectable reporter sequences (e.g., antibiotic resistance genes), origins of replication, and the like. The nucleic acid constructs can further comprise RNA processing elements such as glycine tRNAs, or Csy4 recognition sites. Such RNA processing elements can, for instance, intersperse polynucleotide sequences encoding multiple gRNAs under the control of a single promoter to produce the multiple gRNAs from a transcript encoding the multiple gRNAs. When a cys4 recognition cite is used, a vector can further comprise sequences for expression of Csy4 RNAse to process the gRNA transcript. Additional information about nucleic acid constructs and use thereof may be found in “Current Protocols in Molecular Biology”, Ausubel et al., John Wiley & Sons, New York, 2003, or “Molecular Cloning: A Laboratory Manual”, Sambrook & Russell, Cold Spring Harbor Press, Cold Spring Harbor, NY, 3rd edition, 2001. Other methods of controlling expression in a specific tissue or target cell can be as described in Sections I(d) and IV (a).

Nucleic acid constructs encoding an expression modification system can comprise one or more constructs encoding the expression system. The nucleic acid constructs can be DNA or RNA, linear or circular, single-stranded or double-stranded, or any combination thereof. The nucleic acid constructs can be codon optimized for efficient translation into protein in the cell of interest. Codon optimization programs are available as freeware or from commercial sources.

In some aspects, the protein expression system comprises a nucleic acid sequence encoding ACMSD operably linked to at least one promoter control sequence. In some aspects, the nucleic acid sequence encoding ACMSD comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 5. In some aspects, the nucleic acid sequence encoding ACMSD comprises about 75% or more, 85% or more, 95% or more, or 100% sequence identity with SEQ ID NO: 5.

In some aspects, the expression system encodes an mRNA comprising a nucleic acid sequence encoding ACMSD, wherein the nucleic acid sequence of the mRNA comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 4. In some aspects, the expression system encodes an mRNA comprising a nucleic acid sequence encoding ACMSD, wherein the nucleic acid sequence of the mRNA comprises about 75% or more, 85% or more, 95% or more, or 100% sequence identity with SEQ ID NO: 4.

In some aspects, the at least one promoter control sequence is a tissue or cell-specific promoter control sequence. For instance, when the cells or tissues are in the central nervous system, the protein modification system can comprise a nucleic acid sequence encoding ACMSD operably linked to at least one promoter control sequence is an interneuron specific promoter, a hippocampal promoter, a dopamine beta-hydroxylase promoter, a glutamatergic neuron promoter, a tyrosine hydroxylase promoter, a motor neuron promoter, a serotonergic promoter, a microglial promoter, an astrocyte specific promoter, an oligodendrocyte specific promoter, or any combination thereof. In some aspects, the promoter is CD38, F4/80, GFAP for glial subtypes, CMV and truncated CAG ubiquitous promoter, or any combination thereof.

In some aspects, the expression modification system comprises DNA coding sequences operably linked to at least one ubiquitous promoter control sequence. In some aspects, the ubiquitous promoter control sequence is the chicken beta actin(CBA)/cytomegalovirus (CMV) promoter hybrid. In some aspects, the ubiquitous promoter control sequence is the CBA/CMV promoter hybrid. In some aspects, the expression modification system comprises a nucleic acid sequence encoding ACMSD operably linked to at least one ubiquitous promoter control sequence. In some aspects, the expression modification system comprises a nucleic acid sequence encoding ACMSD operably linked to the CBA/CMV promoter hybrid. In some aspects, the nucleic acid sequence of the CBA/CMV promoter hybrid comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 7. In some aspects, the nucleic acid sequence of the CBA/CMV promoter hybrid comprises about 75% or more, 85% or more, 95% or more, or 100% sequence identity with SEQ ID NO: 7.

In some aspects, the nucleic acid sequence of the ACMSD expression construct comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 8. In some aspects, the nucleic acid sequence of the ACMSD expression construct comprises about 75% or more, 85% or more, 95% or more, or 100% sequence identity with SEQ ID NO: 8.

In some aspects, the protein expression modification system comprises a nucleic acid expression construct for expressing an shRNA sequence targeting a sequence within a nucleic acid sequence encoding the ACMSD protein, wherein the expression construct comprises a nucleotide sequence encoding an shRNA molecule operably linked to at least one promoter. In some aspects, the at least one promoter control sequence is a tissue or cell-specific promoter control sequence. In some aspects, the promoter comprises a nucleotide sequence encoding a human H1 polymerase III promoter such as a promoter having a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 16. In some aspects, the promoter comprises a nucleotide sequence encoding a human H1 polymerase III promoter such as a promoter comprising a nucleic acid sequence having about 75% or more, 85% or more, 95% or more, or 100% sequence identity with SEQ ID NO: 16.

### III. Libraries

A further aspect of the present disclosure provides a library of engineered systems comprising a plurality of engineered systems for expressing ACMSD. As used herein, the term “library” refers to a collection of entities, such as, for example, engineered systems. A library can comprise at least two, at least three, at least four, at least five, at least ten, at least 25, at least 50, at least 102, at least 103, at least 104, at least 105, at least 106, at least 107, at least 108, at least 109, or more different entities (e.g., peptides, nucleic acids). Libraries provided herein comprise a plurality of library members and libraries of nucleic acid compositions each encoding a member of the library.

Each of the plurality of engineered systems can increase expression of ACMSD ubiquitously, or in a specific target cell or tissue. In some aspects, the nucleic acid delivery system is an rAAV vector. Accordingly, an aspect of the instant disclosure is a library of rAAVs comprising a plurality of rAAV members, each comprising a nucleic acid construct encoding an ACMSD protein expression system. rAAV vectors suitable for the instant disclosure can be as described in International Patent Application No. PCT/US2021/023314, the entire disclosure of which is incorporated herein by reference.

As explained above, increased expression of ACMSD can be targeted to a specific cell or tissue type by using protein expression modification systems with tissue-specific control sequences, using delivery systems that can, but do not necessarily, have tropism to a desired target cell or tissue type for delivering the expression systems to the target cell or tissue type, and combinations therefor.

In some aspects, the library comprises a plurality of engineered systems for expressing ACMSD, wherein each of the plurality of engineered systems comprises a non-cell or tissue-specific delivery system, a plurality of cell-or tissue-specific ACMSD expression systems each targeted to express ACMSD in a specific target cell, or any combination thereof. Cell or non-cell specific ACMSD expression systems expressing various levels of ACMSD are also envisioned. In one aspect, the engineered system comprises non-cell specific rAAV vector. In one aspect, the non-cell rAAV vector comprises an AAV2 capsids comprising the Y444F, Y500F, Y730F, T491V, R585S, R588T, R487G amino acid substitutions, or corresponding substitutions in the capsid protein of another AAV serotype in various combinations and/or in combination with incorporation of mutations that enhance tropism of the virus to a desired target cell or tissue type.

In some aspects, the library comprises a plurality of engineered systems for expressing ACMSD, wherein each of the plurality of engineered systems comprises a plurality of cell or tissue-specific delivery systems, such as viral vectors having tropism to specific tissues or cells, and a protein expression system for ubiquitous expression of ACMSD. In one aspect, the cell or tissue-specific delivery system is an rAAV vector having tropism to a target cell or tissue.

In some aspects, the library comprises a plurality of engineered systems for expressing ACMSD, wherein each of the plurality of engineered systems comprises a plurality of cell or tissue-specific delivery systems, and a plurality of cell-or tissue-specific ACMSD expression systems.

In all of the previous aspects, the target cell or tissue can be a cell of the nervous system.

### IV. Methods

Another aspect of the present disclosure encompasses a method of providing a protective effect, therapeutic effect, or any combination thereof to a subject in need thereof. Providing a protective effect can prevent, delay, or arrest development of the disease condition in the subject. The method comprises administering a therapeutically effective amount of an engineered system for modifying the expression of ACMSD protein in a target cell. The engineered system can be as described above in Section I.

The subject can be a human, a livestock animal, a companion animal, a lab animal, or a zoological animal. In one aspect, the subject can be a rodent, e.g. a mouse, a rat, a guinea pig, etc. Non-limiting examples of suitable livestock animals can include pigs, cows, horses, goats, sheep, llamas and alpacas. Non-limiting examples of companion animals can include pets such as dogs, cats, rabbits, and birds. As used herein, a “zoological animal” refers to an animal that can be found in a zoo. Such animals can include non-human primates, large cats, wolves, and bears. Non-limiting examples of a laboratory animal can include rodents, canines, felines, and non-human primates. Non-limiting examples of rodents can include mice, rats, guinea pigs, etc. In some aspects, the subject is a human subject.

In some aspects, the method comprises expressing ACMSD in a specific tissue or target cell. Expression of ACMSD in specific target cells or tissues can be as described in Section II. Other methods of expressing ACMSD in specific cells or tissues can be as described in Section IV(a) below. In some aspects, the method comprises expressing ACMSD in the central nervous system. In some aspects, the method comprises expressing ACMSD in microglia. In some aspects, the method comprises expressing ACMSD in astrocytes. The expression can also comprise expressing ACMSD ubiquitously in any cell or tissue type. Non-cell or tissue-specific expression of ACMSD can be as described in Section II.

The method comprises treating a disease condition associated with inflammation, oxidative stress, protein aggregation, energy failure, and toxic exposure such as exposure to pollutants. In some aspects, the method comprises treating a neurological condition. In some aspects, the method comprises treating a neurological condition caused by inflammation. In some aspects, the method comprises treating Parkinson's disease, Alzheimer's disease, ALS, Huntington's disease, brain ischemia, CNS infections and autoimmune disorders, mood disorders, schizophrenia, or any combination thereof.

It will be appreciated by those skilled in the art that a combination of more than one expression construct or vector-mediated system of the present disclosure can be used. It will also be appreciated by those skilled in the art that an engineered system of the present disclosure can be used in combination with other therapeutic agents before, after, and/or during treatment with an engineered system of the disclosure. Further, methods of the invention can be used in combination with standard treatments for a specific disorder.

It will also be appreciated that the method can further comprise specifically targeting expression of the ACMSD protein into specific cell types and tissues using methods known by individuals of skill in the art. Non-limiting examples of methods of targeting expression of a protein such as ACMSD into specific tissues or cell types include use of tissue or cell-specific promoters, vectors having tropism to specific tissues or cell types, or use of a de-targeting nucleic acid sequence. A de-targeting nucleic acid sequence is a nucleic acid sequence that, when expressed in a cell type, will prevent or reduce expression of a nucleic acid sequence in that cell type. For instance, if the vector is an AAV vector, expression of ACMSD in neurons can be prevented by detargeting the AAV vector by, e.g., including MIR124 binding sites.

The programmable nucleic acid modification system can be an interfering nucleic acid molecule, a de-targeting nucleic acid sequence, or a nucleic acid editing system. A de-targeting nucleic acid sequence is a nucleic acid sequence that, when expressed in a cell type, will prevent or reduce expression of a nucleic acid sequence in that cell type. For instance, if

### (a) Administering

An engineered system of the invention can be formulated and administered to a subject by several different means. Methods of administration can also be used to provide selective expression in target cells or tissues by delivering the engineered system specifically to the target cell of tissue. For instance, when a method of the instant disclosure comprises providing a protective or therapeutic effect against Huntington's disease, an expression system of the instant disclosure can be administered by direct injection of the expression system into the basal ganglia; when a method of the instant disclosure comprises providing a protective or therapeutic effect against Parkinson's disease, an expression system of the instant disclosure can be administered by direct injection of the expression system into the substantia nigra, and when a method of the instant disclosure comprises providing a protective or therapeutic effect against Alzheimer's disease, an expression system of the instant disclosure can be administered by direct injection of the expression system into the hippocampus.

An engineered system can generally be administered parenterally, intraperitoneally, intravascularly, transdermally, subcutaneously, rectally, or intrapulmonarily in dosage unit formulations containing conventional nontoxic pharmaceutically acceptable adjuvants, carriers, excipients, and vehicles as desired. The term parenteral as used herein includes subcutaneous, intravenous, intramuscular, intraparenchymal, intrathecal, or intracisternal injection, or infusion techniques. Formulation of pharmaceutical systems is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

When the delivery system is a non-viral construct, and/or vector to introduce the programmable nucleic acid modification system into a cell or organism, the vectors can be delivered to a cell or tissue by electroporation, using a variety of means. Suitable delivery means include synthetic oligonucleotides, lipoplexes polymersomes, polyplexes, dendrimers, inorganic nanoparticles, cell-penetrating peptides, microinjection, electroporation, sonoporation, biolistics, calcium phosphate-mediated transfection, cationic transfection, liposomes and other lipids, dendrimer transfection, heat shock transfection, nucleofection transfection, gene gun delivery, dip transformation, supercharged proteins, cell-penetrating peptides, implantable devices, magnetofection, lipofection, impalefection, optical transfection, proprietary agent-enhanced uptake of nucleic acids, proprietary agent-enhanced uptake of nucleic acids, and delivery via liposomes, immunoliposomes, virosomes, or artificial virions.

When the delivery system is a viral vector, cells can be infected with the viral vector by contacting the cells with the vector. For instance, the cells can be tissue culture cells, and can be contacted with the viral vector by adding the vector to the cell culture. The cells can also be infected by delivering to a subject in compositions according to any appropriate methods known in the art. The viral vector, suspended in a physiologically compatible carrier (e.g., in a composition), can be administered to a subject, e.g., host animal, such as a human, mouse, rat, cat, dog, sheep, rabbit, horse, cow, goat, pig, guinea pig, hamster, chicken, turkey, or a non-human primate (e.g., Macaque).

Delivery of the engineered system to a mammalian subject can be by, for example, intramuscular injection or by administration into the bloodstream of the mammalian subject. Administration into the bloodstream can be by injection into a vein, an artery, or any other vascular conduit. In some aspects, the engineered systems are administered into the bloodstream by way of isolated limb perfusion, a technique well known in the surgical arts, the method essentially enabling the artisan to isolate a limb from the systemic circulation prior to administration of the engineered system. A variant of the isolated limb perfusion technique can also be employed by the skilled artisan to administer the engineered systems into the vasculature of an isolated limb to potentially enhance transduction into muscle cells or tissue. Moreover, in certain aspects, it can be desirable to deliver the engineered systems to the nervous system of a subject. Thus, the term includes, but is not limited to, neuronal cells, glial cells, astrocytes, cerebrospinal fluid (CSF), interstitial spaces, bone, cartilage and the like. engineered systems can be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection.

Suitable carriers can be readily selected by one of skill in the art in view of the indication for which an engineered system is directed. For example, one suitable carrier includes saline, which can be formulated with a variety of buffering solutions (e.g., phosphate buffered saline). Other carriers include sterile saline, lactose, sucrose, calcium phosphate, gelatin, dextran, agar, pectin, peanut oil, sesame oil, and water. The selection of the carrier is not a limitation of the disclosure.

Optionally, in addition to the delivery system and carrier(s), other conventional pharmaceutical ingredients can be included, such as preservatives or chemical stabilizers. Suitable preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

Delivery systems are administered in sufficient amounts to introduce the nucleic acid construct into the cells of a desired tissue and to provide sufficient levels of expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration can be combined, if desired.

Formulation of pharmaceutically-acceptable excipients and carrier solutions is well known to those of skill in the art, as is the development of suitable dosing and treatment regimens for using the particular systems described herein in a variety of treatment regimens. The amount of active compound in each therapeutically-useful composition can be prepared is such a way that a suitable dosage is obtained in any given unit dose of the compound. Factors such as solubility, bioavailability, biological half-life, route of administration, product shelf life, as well as other pharmacological considerations, are contemplated by one skilled in the art of preparing such pharmaceutical formulations, and as such, a variety of dosages and treatment regimens can be desirable.

In certain aspects, when the delivery system is a viral vector, it is desirable to deliver the delivery system in suitably formulated pharmaceutical compositions disclosed herein either subcutaneously, intrapancreatically, intranasally, parenterally, intravenously, intramuscularly, intrathecally, or orally, intraperitoneally, or by inhalation. In some aspects, the cells are infected with the viral vectors by administering the vectors to a subject in a pharmaceutically-acceptable carrier to the subject in an amount and for a period of time sufficient to infect the cells. For instance, the viral vectors can be administered parenterally into the subject.

Pharmaceutical forms suitable for injectable use can include sterile aqueous solutions or dispersions and sterile powders for the extemporaneous preparation of sterile injectable solutions or dispersions. Dispersions can also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, isotonic agents can be included, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution can be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, sterile aqueous mediums that can be employed are known to those of skill in the art.

Sterile injectable solutions can be prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the other required ingredients from those enumerated above. In the case of sterile powders for the preparation of sterile injectable solutions, the methods of preparation can be vacuum-drying and freeze-drying techniques which yield a powder of the active ingredient, plus any additional desired ingredient from a previously sterile-filtered solution thereof.

Engineered systems can also be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions are administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles and the like, can be used for the introduction of the compositions of the disclosure into suitable host cells. In particular, the engineered system can be formulated for delivery either encapsulated in a lipid particle, a liposome, a vesicle, a nanosphere, or a nanoparticle or the like.

### (b) Disease Condition

As explained herein above, expression of ACMSD can support growth and development, survival, and differentiation of cells, provide protection from inflammation, oxidative stress, protein aggregation, cellular organelle dysfunction, energy failure, and toxic exposure such as exposure to pollutants, and can treat and prevent related and other health conditions. The disease conditions can be induced by external factors or the subject can be genetically predisposed to acquire a condition. Accordingly, methods of the instant disclosure can be used to treat any disease conditions that can benefit from expression of ACMSD, including disease conditions associated with inflammation, oxidative stress, protein aggregation, energy failure, toxic exposure such as exposure to pollutants, genetic factors, environmental conditions, immune system activity, or factors not currently identified. Despite the fact that at least some aspect of the pathology of each of the diseases mentioned above is different from the other diseases, their pathologies ordinarily share other features, so that they can be considered as a group. Furthermore, aspects of their pathologies that they have in common often make it possible to treat them with similar therapeutic methods.

Methods of the instant disclosure can provide a general protective effect, or a protective effect against disease conditions caused by specific conditions such as, e.g., inflammation, oxidative stress, protein aggregation, energy failure, and toxic exposure such as exposure to pollutants. The disease condition can be associated with, or related to the expression of ACMSD, or can be a disease condition not having a direct link between ACMSD and a specific disease condition. The methods can provide protection from diseases conditions even when development of the condition is not suspected or anticipated.

In some aspects, the method comprises providing neurotrophic factor activity in the nervous tissue of a subject. In some aspects, the method comprises providing a neuroprotective environment in the nervous system. In some aspects, the method comprises providing an anti-inflammatory effect in the nervous tissue. In some aspects, the method comprises providing a protective effect against a neurological condition. The neurological condition can be Parkinson's disease, Alzheimer's disease, ALS, Huntington's disease, brain ischemia, CNS infections, and autoimmune disorders. In some aspects, the method comprises providing a protective effect against a psychiatric condition or disorder. No-limiting examples of psychiatric disorders include mood disorders including suicidal thoughts, suicidality, and violent suicide, ADHD, schizophrenia, OCD, autism, anxiety, addiction/substance abuse, bipolar disorder, various forms of depression, and post-traumatic stress disorder, more commonly known as PTSD.

### A. Inflammation

In some aspects, the disease condition is inflammation or a condition associated with inflammation. Inflammation can be in any cell type of any tissue or organ. For instance, inflammation in the eye can be in rod photoreceptor cells, cone photoreceptor cells, retinal pigment epithelial (RPE) cells. Non-limiting examples of disorders associated with, caused by, or resulting from inflammation include Inflammatory disorders include encephalitis, myelitis, meningitis; arachnoiditis; neuritis; dacryoadenitis; scleritis; episcleritis; keratitis; retinitis; chorioretinitis; blepharitis; conjunctivitis; uveitis; otitis externa; otitis media; labyrinthitis; mastoiditis; carditis; endocarditis; myocarditis; pericarditis; vasculitis; arteritis; phlebitis; capillaritis; sinusitis; rhinitis; allergic rhinitis, pharyngitis; laryngitis; tracheitis; bronchitis; pneumonitis; pleuritis; mediastinitis; stomatitis; gingivitis; gingivostomatitis; glossitis; tonsillitis; sialadenitis/parotitis; cheilitis; pulpitis; gnathitis; esophagitis; gastritis; gastroenteritis; enteritis; gastrointestinal conditions such as inflammatory bowel disease, Crohn's disease, gastritis, irritable bowel syndrome, or ulcerative colitis; colitis; enterocolitis; duodenitis; ileitis; caecitis; appendicitis; proctitis; hepatitis; ascending cholangitis; cholecystitis; pancreatitis; peritonitis; folliculitis; cellulitis; hidradenitis; dermatomyositis; myositis; synovitis/tenosynovitis; enthesitis; fasciitis; capsulitis; epicondylitis; tendinitis; panniculitis; osteochondritis: osteitis/osteomyelitis; spondylitis; periostitis; chondritis; nephritis; glomerulonephritis; pyelonephritis; ureteritis; cystitis; urethritis; oophoritis; salpingitis; endometritis; parametritis; cervicitis; vaginitis; vulvitis; mastitis; orchitis; epididymitis; prostatitis; seminal vesiculitis; balanitis; posthitis; balanoposthitis; chorioamnionitis; funisitis; omphalitis; insulitis; hypophysitis; thyroiditis; parathyroiditis; adrenalitis; lymphangitis; lymphadenitis, arthritic conditions including, but not limited to, rheumatoid arthritis, spondyloarthropathies, gouty arthritis, osteoarthritis, systemic lupus erythematosus, or juvenile arthritis, asthma, bronchitis, menstrual cramps, premature labor, bursitis, skin-related conditions such as psoriasis, eczema, burns, dermatitis, vascular diseases such as migraine headaches, periarteritis nodosa, aplastic anemia, Hodgkin's disease, scleroderma, rheumatic fever, type I diabetes, neuromuscular junction disease including myasthenia gravis, white matter disease, multiple sclerosis, sarcoidosis, nephrotic syndrome, Behcet's syndrome, polymyositis, hypersensitivity, respiratory distress syndrome, endotoxin shock syndrome, atherosclerosis, cancers, conditions associated with pulmonary inflammation, such as that associated with viral infections or cystic fibrosis, neuroinflammatory diseases such as Alzheimer's disease (AD), stroke, traumatic brain injury (TBI), Parkinson's disease (PD), Amyotrophic lateral sclerosis (ALS), multiple sclerosis (MS), ataxia, Bell's palsy, or epilepsy.

ix. Acute Inflammation

In some aspects, a method of the instant disclosure comprises treating acute inflammation. Acute inflammation occurs immediately upon injury, lasting only a few days. Cytokines and chemokines promote the migration of neutrophils and macrophages to the site of inflammation. Pathogens, allergens, toxins, burns, and frostbite are some of the typical causes of acute inflammation. Acute inflammation can be a defensive mechanism to protect tissues against injury. Inflammation lasting 2-6 weeks is designated subacute inflammation. Acute inflammation may be regarded as the first line of defense against injury. Acute inflammatory response requires constant stimulation to be sustained. Inflammatory mediators are short-lived and are quickly degraded in the tissue. Hence, acute inflammation begins to cease once the stimulus has been removed. Acute inflammation can be categorized as mild, moderate, severe, and systemic severe inflammation. Symptoms of mild, moderate, severe, and systemic severe inflammation can and will vary depending on the type of inflammation and can be as recognized in the art for each type of inflammation. For instance, a mild and localized bacterial infection on the skin can develop into a systemic severe inflammation during sepsis.

Acute inflammation is a short-term process, usually appearing within a few minutes or hours and begins to cease upon the removal of the injurious stimulus. It involves a coordinated and systemic mobilization response locally of various immune, endocrine, and neurological mediators of acute inflammation. In a normal healthy response, it becomes activated, clears the pathogen or source of injury, begins a repair process, and then ceases. It is characterized by five cardinal signs: pain, calor heat, redness, swelling, and loss of function. Redness and heat are due to increased blood flow at body core temperature to the inflamed site; swelling is caused by accumulation of fluid; and pain is due to the release of chemicals such as bradykinin and histamine that stimulate nerve endings. Loss of function has multiple causes.

However, an infectious organism can escape the confines of the immediate tissue via the circulatory system or lymphatic system, where it may spread to other parts of the body to cause severe acute inflammation. If an organism is not contained by the actions of acute inflammation, it may gain access to the lymphatic system via nearby lymph vessels. An infection of the lymph vessels is known as lymphangitis, and infection of a lymph node is known as lymphadenitis. When lymph nodes cannot destroy all pathogens, the infection spreads further. A pathogen can gain access to the bloodstream through lymphatic drainage into the circulatory system. When inflammation overwhelms the host, systemic inflammatory response syndrome is diagnosed. When it is due to infection, the term sepsis is applied, with the term's bacteremia being applied specifically for bacterial sepsis and viremia specifically to viral sepsis as is the case with COVID-19. Vasodilation and organ dysfunction are serious problems associated with widespread infection that may lead to septic shock and death.

x. Chronic Inflammation

In some aspects, a method of the instant disclosure comprises treating chronic inflammation. Chronic systemic inflammation (SI) is the result of release of pro-inflammatory cytokines from immune-related cells and the chronic activation of the innate immune system. Chronic inflammation can last for months or years. Macrophages, lymphocytes, and plasma cells predominate in chronic inflammation, in contrast to the neutrophils that predominate in acute inflammation. Chronic systemic inflammation can contribute to the development or progression of certain conditions such as cardiovascular disease, cancer, diabetes mellitus, chronic kidney disease, non-alcoholic fatty liver disease, allergies, autoimmune and neurodegenerative disorders, coronary heart disease, and chronic obstructive pulmonary disease (COPD) are examples of diseases mediated by chronic inflammation. Obesity, smoking, stress and insufficient diet are some of the factors that promote chronic inflammation. Common signs and symptoms that develop during chronic inflammation include body pain, arthralgia, myalgia, chronic fatigue and insomnia, depression, anxiety, and mood disorders, gastrointestinal complications such as constipation, diarrhea, and acid reflux, weight gain or loss, and frequent infections.

### B. Cancer

In some aspects, the disease condition is cancer or a neoplasm. The neoplasm can be malignant or benign, the cancer can be primary or metastatic; the neoplasm or cancer can be early stage or late stage. Non-limiting examples of neoplasms or cancers that can be treated include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sézary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), enknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, and Wilms tumor (childhood).

In other aspects, the disease condition is an immune system condition. Non-limiting examples include diseases associated with a weak immune system (primary immune deficiency), a disease associated with a weakened system (acquired immune deficiency), an immune system that is too active (allergic reactions), or an autoimmune disease. Non-liming examples of immune system conditions include severe combined immunodeficiency (SCID), rheumatoid arthritis, osteoarthritis, Chron's disease, angiofibroma, ocular diseases (e.g., retinal vascularisation, diabetic retinopathy, age-related macular degeneration, macular degeneration, etc.), obesity, Alzheimer's disease, restenosis, autoimmune diseases, allergy, asthma, endometriosis, atherosclerosis, vein graft stenosis, peri-anastomatic prothetic graft stenosis, prostate hyperplasia, chronic obstructive pulmonary disease, psoriasis, inhibition of neurological damage due to tissue repair, scar tissue formation (and can aid in wound healing), multiple sclerosis, inflammatory bowel disease, infections, particularly bacterial, viral, retroviral or parasitic infections (by increasing apoptosis), pulmonary disease, neoplasm, Parkinson's disease, transplant rejection (as an immunosuppressant), septic shock, etc.

In some aspects, the disease condition is a neurological disease condition or disorder. Neurological diseases can be neurodegenerative diseases. Neurodegenerative diseases result from the deterioration of neurons, causing brain dysfunction. The diseases are loosely divided into two groups—conditions affecting memory that are ordinarily related to dementia and conditions causing problems with movements. The most widely known neurodegenerative diseases include Alzheimer (or Alzheimer's) disease and its precursor mild cognitive impairment (MCI), Parkinson's disease (including Parkinson's disease dementia), and multiple sclerosis.

Less well-known neurodegenerative diseases include adrenoleukodystrophy, AIDS dementia complex, Alexander disease, Alper's disease, amyotrophic lateral sclerosis (ALS), ataxia telangiectasia, Batten disease, bovine spongiform encephalopathy, Canavan disease, cerebral amyloid angiopathy, cerebellar ataxia, Cockayne syndrome, corticobasal degeneration, Creutzfeldt-Jakob disease, diffuse myelinoclastic sclerosis, fatal familial insomnia, Fazio-Londe disease, Friedreich's ataxia, frontotemporal dementia or lobar degeneration, hereditary spastic paraplegia, Huntington disease, Kennedy's disease, Krabbe disease, Lewy body dementia, Lyme disease, Machado-Joseph disease, motor neuron disease, Multiple systems atrophy, neuroacanthocytosis, Niemann-Pick disease, Pelizaeus-Merzbacher Disease, Pick's disease, primary lateral sclerosis including its juvenile form, progressive bulbar palsy, progressive supranuclear palsy, Refsum's disease including its infantile form, Sandhoff disease, Schilder's disease, spinal muscular atrophy, spinocerebellar ataxia, Steele-Richardson-Olszewski disease, subacute combined degeneration of the spinal cord, survival motor neuron spinal muscular atrophy, Tabes dorsalis, Tay-Sachs disease, toxic encephalopathy, transmissible spongiform encephalopathy, Vascular dementia, and X-linked spinal muscular atrophy, as well as idiopathic or cryptogenic diseases as follows: synucleinopathy, progranulinopathy, tauopathy, amyloid disease, prion disease, protein aggregation disease, and movement disorder. A more comprehensive listing can be found at the web site (www) of the National Institute of Neurological Disorders and Stroke (ninds) of the National Institutes of Health (nih) of the United States government (gov) in a subdirectory (/disorder/disorder_index) web page (htm). It is understood that such diseases often go by more than one name and that a nosology can oversimplify pathologies that occur in combination or that are not archetypical. In some aspects, the neurological condition Alzheimer's disease (AD), Parkinson's disease (PD), Amyotrophic lateral sclerosis (ALS), multiple sclerosis (MS), ataxia, Bell's palsy, and epilepsy CNS infections and autoimmune disorders, mood disorders such as suicidality and violent suicide, schizophrenia, or any combination thereof.

Certain other disorders, such as postoperative cognitive dysfunction have been described only recently, and they too can involve neuro-degeneration. Other disorders such as epilepsy cannot be primarily neurodegenerative, but at some point in their progression, they might involve nerve degeneration.

### V. Kits

A further aspect of the present disclosure provides kits comprising one or more engineered system, one or more nucleic acid construct encoding the engineered system or the nucleic acid construct encoding an ACMSD protein expression modification system, or one or more library of engineered systems for expressing ACMSD. Engineered systems can be as described in Section I above, nucleic acid constructs encoding the engineered system or the nucleic acid construct encoding an ACMSD protein expression modification system can be as described in Section II, and libraries of engineered systems for expressing ACMSD can be as described in Section III above. Alternatively, the kit can comprise one or more cells comprising one or more engineered system, one or more nucleic acid construct encoding the engineered system or the nucleic acid construct encoding an ACMSD protein expression modification system, one or more library of engineered systems for expressing ACMSD, or any combination thereof

The kits can further comprise transfection reagents, cell growth media, selection media, in vitro transcription reagents, nucleic acid purification reagents, protein purification reagents, buffers, and the like. The kits provided herein generally include instructions for carrying out the methods detailed below. Instructions included in the kits can be affixed to packaging material or can be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there can be additional elements other than the listed elements.

The term “a therapeutically effective amount” as used herein refers to an amount effective, at dosages, and for periods of time necessary, to achieve a desired general protective effect, and the desired result with respect to the treatment of a disease or protection from a disease. For example, in the protection from a neurodegenerative disease, an agent (i.e., a compound or a composition) which protects from, decreases, prevents, delays, or suppresses or arrests any symptoms of the neurodegenerative disease would be effective. An effective amount of an agent is not required to cure a disease or condition but will provide a treatment for a disease or condition such that the onset of the disease or condition is delayed, hindered or prevented, or the disease or condition symptoms are ameliorated. The effective amount can be divided into one, two or more doses in a suitable form to be administered at one, two or more times throughout a designated time period. A therapeutically effective amount can be determined by the efficacy or potency of the particular composition, the disorder being treated, the duration or frequency of administration, the method of administration, and the size and condition of the subject, including that subject's particular treatment response. A therapeutically effective amount can be determined using methods known in the art, and can be determined experimentally, derived from therapeutically effective amounts determined in model animals such as the mouse, or a combination thereof. Additionally, the route of administration can be considered when determining the therapeutically effective amount. In determining therapeutically effective amounts, one skilled in the art can also consider the existence, nature, and extent of any adverse effects that accompany the administration of a particular compound in a particular subject.

As used herein, the terms “to cure,” “curative effect,” “treating,” “treatment,” “to treat” can be used interchangeably and each can mean to protect from, alleviate, suppress, repress, eliminate, prevent or slow the appearance of symptoms, clinical signs, or underlying pathology of a condition or disorder on a temporary or permanent basis. Protection of cells or tissues involves administering an agent of the present invention to a subject prior to onset of a condition, even when development of the condition is not suspected. Treating a condition or disorder involves administering an agent of the present invention to a subject prior to onset of the condition. Suppressing a condition or disorder involves administering an agent of the present invention to a subject after induction of the condition or disorder but before its clinical appearance. Repressing the condition or disorder involves administering an agent of the present invention to a subject after clinical appearance of the disease. Prophylactic treatment can reduce the risk of developing the condition and/or lessen its severity if the condition later develops. For instance, treatment of a microbial infection can reduce, ameliorate, or altogether eliminate the infection, or prevent it from worsening.

As used herein, the terms “treatment” or “treating” refers to arresting, inhibiting, correcting, or attempting to arrest or inhibit or correct, the existence, development, or progression of a disorder and/or causing, or attempting to cause, the reduction, suppression, regression, or remission of a disorder and/or a symptom thereof. As would be understood by those skilled in the art, various clinical and scientific methodologies and assays may be used to assess the development or progression of a disorder, and similarly, various clinical and scientific methodologies and assays may be used to assess the reduction, regression, or remission of a disorder or its symptoms.

As used herein, the term “treating” refers to: (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures. In the context of a neurodegenerative disorder, “treat” and “treating” encompass alleviating, ameliorating, delaying the onset of, inhibiting the progression of, or reducing the severity of one or more symptoms associated with the neurodegenerative disorder.

As used herein, the term neurotrophic effect refers to an effect provided by neurotrophic factors. Neurotrophic factors are biomolecules that support the growth, survival, and differentiation of both developing and mature neurons.

As used herein, the terms “medical condition” and “disease condition” are used interchangeable and include, but are not limited to, any condition or disease manifested as one or more physical and/or psychiatric symptoms for which treatment is desirable, and includes previously and newly identified diseases and other disorders.

The term “subject” can refer to a human, a livestock animal, a companion animal, a lab animal, or a zoological animal. The subject can be a rodent, e.g. a mouse, a rat, a guinea pig, etc. Non-limiting examples of suitable livestock animals can include pigs, cows, horses, goats, sheep, llamas and alpacas. Non-limiting examples of companion animals can include pets such as dogs, cats, rabbits, and birds. As used herein, a “zoological animal” refers to an animal that can be found in a zoo. Such animals can include non-human primates, large cats, wolves, and bears. Non-limiting examples of a laboratory animal can include rodents, canines, felines, and non-human primates. Non-limiting examples of rodents can include mice, rats, guinea pigs, etc.

As used herein, the administration of an agent or drug to a subject or patient includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

A “genetically modified” cell refers to a cell in which the nuclear, organellar or extrachromosomal nucleic acid sequences of a cell has been modified, i.e., the cell contains at least one nucleic acid sequence that has been engineered to contain an insertion of at least one nucleotide, a deletion of at least one nucleotide, and/or a substitution of at least one nucleotide.

As used herein, the term “gene” means a segment of DNA that contains all the information for the regulated biosynthesis of an RNA product, including promoters, exons, introns, and other untranslated regions that control expression.

As used herein, “expression” includes but is not limited to one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

The terms “genome modification” and “genome editing” refer to processes by which a specific nucleic acid sequence in a genome is changed such that the nucleic acid sequence is modified. The nucleic acid sequence can be modified to comprise an insertion of at least one nucleotide, a deletion of at least one nucleotide, and/or a substitution of at least one nucleotide. The modified nucleic acid sequence is inactivated such that no product is made. Alternatively, the nucleic acid sequence can be modified such that an altered product is made.

As used herein, the term “mutant” means any heritable variation from the wild-type that is the result of a mutation, e.g., single nucleotide polymorphism (“SNP”). The term “mutant” is used interchangeably with the terms “marker”, “biomarker”, and “target” throughout the specification.

The terms “nucleic acid” and “polynucleotide” refer to a deoxyribonucleotide or ribonucleotide polymer, in linear or circular conformation. For the purposes of the present disclosure, these terms are not to be construed as limiting with respect to the length of a polymer. The terms can encompass known analogs of natural nucleotides, as well as nucleotides that are modified in the base, sugar and/or phosphate moieties. In general, an analog of a particular nucleotide has the same base-pairing specificity, i.e., an analog of A will base-pair with T. The nucleotides of a nucleic acid or polynucleotide can be linked by phosphodiester, phosphothioate, phosphoramidite, phosphorodiamidate bonds, or any combination thereof.

The term “nucleotide” refers to deoxyribonucleotides or ribonucleotides. The nucleotides can be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog can be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

As used herein, the term “polypeptide” means any polypeptide comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well-known in the art. Such modifications are well described in basic texts and in more detailed monographs, as well as in a voluminous research literature.

As used herein, the terms “target site”, “target sequence,” or “nucleic acid locus” refer to a nucleic acid sequence that defines a portion of a nucleic acid sequence to be modified or edited and to which a homologous recombination composition is engineered to target.

The terms “upstream” and “downstream” refer to locations in a nucleic acid sequence relative to a fixed position. Upstream refers to the region that is 5′ (i.e., near the 5′ end of the strand) to the position, and downstream refers to the region that is 3′ (i.e., near the 3′ end of the strand) to the position.

As various changes could be made in the above-described cells and methods without departing from the scope of the invention, it is intended that all matter contained in the above description and in the examples given below, shall be interpreted as illustrative and not in a limiting sense.

SEQUENCES

| SEQ | | |
|---|---|---|
| ID | | |
| NO: | Sequence | Description |
| 1 | ttggccactccctctctgcg | ACMSD shRNA AAV |
| | cgctcgctcgctcactgagg | vector |
| | ccgggcgaccaaaggtcgcc | |
| | cgacgcccgggctttgcccg | |
| | ggcggcctcagtgagcgagc | |
| | gagcgcgcagagagggagtg | |
| | gccaactccatcactagggg | |
| | ttcctagatctgaattcggt | |
| | gctagcaccctagttattaa | |
| | tagtaatcaattacggggtc | |
| | attagttcatagcccatata | |
| | tggagttccgcgttacataa | |
| | cttacggtaaatggcccgcc | |
| | tggctgaccgcccaacgacc | |
| | cccgcccattgacgtcaata | |
| | atgacgtatgttcccatagt | |
| | aacgccaatagggactttcc | |
| | attgacgtcaatgggtggac | |
| | tatttacggtaaactgccca | |
| | cttggcagtacatcaagtgt | |
| | atcatatgccaagtacgccc | |
| | cctattgacgtcaatgacgg | |
| | taaatggcccgcctggcatt | |
| | atgcccagtacatgacctta | |
| | tgggactttcctacttggca | |
| | gtacatctacgtattagtca | |
| | tcgctattaccatggtcgag | |
| | gtgagccccacgttctgctt | |
| | cactctccccatctcccccc | |
| | cctccccacccccaattttg | |
| | tatttatttattttttaatt | |
| | attttgtgcagcgatggggg | |
| | cggggggggggggggggcgc | |
| | gcgccaggcggggcggggcg | |
| | gggcgaggggcggggcgggg | |
| | cgaggcggagaggtgcggcg | |
| | gcagccaatcagagcggcgc | |
| | gctccgaaagtttcctttta | |
| | tggcgaggcggcggcggcgg | |
| | cggccctataaaaagcgaag | |
| | cgcgcggcgggcgggagtcg | |
| | ctgcgacgctgccttcgccc | |
| | cgtgccccgctccgccgccg | |
| | cctcgcgccgcccgccccgg | |
| | ctctgactgaccgcgttact | |
| | cccacaggtgagcgggcggg | |
| | acggcccttctcctccgggc | |
| | tgtaattagcgcttggttta | |
| | atgacggcttgtttcttttc | |
| | tgtggctgcgtgaaagcctt | |
| | gaggggctccgggagggccc | |
| | tttgtgcgggggggagcggc | |
| | tcggggggtgcgtgcgtgtg | |
| | tgtgtgcgtggggagcgccg | |
| | cgtgcggcccgcgctgcccg | |
| | gcggctgtgagcgctgcggg | |
| | cgcggcgcggggctttgtgc | |
| | gctccgcagtgtgcgcgagg | |
| | ggagcgcggccgggggcggt | |
| | gccccgcggtgcgggggggg | |
| | ctgcgaggggaacaaaggct | |
| | gcgtgcggggtgtgtgcgtg | |
| | ggggggtgagcagggggtgt | |
| | gggcgcggcggtcgggctgt | |
| | aacccccccctgcacccccc | |
| | tccccgagttgctgagcacg | |
| | gcccggcttcgggtgcgggg | |
| | ctccgtacggggcgtggcgc | |
| | ggggctcgccgtgccgggcg | |
| | gggggtggcggcaggtgggg | |
| | gtgccgggcgggcgggggcc | |
| | gcctcgggccggggagggct | |
| | cgggggaggggcgcggcggc | |
| | ccccggagcgccggcggctg | |
| | tcgaggcgcggcgagccgca | |
| | gccattgccttttatggtaa | |
| | tcgtgcgagagggcgcaggg | |
| | acttcctttgtcccaaatct | |
| | gtgcggagccgaaatctggg | |
| | aggcgccgccgcaccccctc | |
| | tagcgggcgcggggcgaagc | |
| | ggtgcggcgccggcaggaag | |
| | gaaatgggcggggagggcct | |
| | tcgtgcgtcgccgcgccgcc | |
| | gtccccttctccctctccag | |
| | cctcggggctgtccgcgggg | |
| | ggacggctgccttcgggggg | |
| | gacggggcagggcggggttc | |
| | ggcttctggcgtgtgaccgg | |
| | cggctctagagcctctgcta | |
| | accatgttcatgccttcttc | |
| | tttttcctacagctcctggg | |
| | caacgtgctggttattgtgc | |
| | tgtctcatcattttggcaaa | |
| | gtattcctcgaagatctgct | |
| | agcaggcgcgcggccgccgc | |
| | caccatgagcaagggcgagg | |
| | aactgttcactggcgtggtc | |
| | ccaattctcgtggaactgga | |
| | tggcgatgtgaatgggcaca | |
| | aattttctgtcagcggagag | |
| | ggtgaaggtgatgccacata | |
| | cggaaagctcaccctgaaat | |
| | tcatctgcaccactggaaag | |
| | ctccctgtgccatggccaac | |
| | actggtcactaccctgacct | |
| | atggcgtgcagtgcttttcc | |
| | agatacccagaccatatgaa | |
| | gcagcatgactttttcaaga | |
| | gcgccatgcccgagggctat | |
| | gtgcaggagagaaccatctt | |
| | tttcaaagatgacgggaact | |
| | acaagacccgcgctgaagtc | |
| | aagttcgaaggtgacaccct | |
| | ggtgaatagaatcgagctga | |
| | agggcattgactttaaggag | |
| | gatggaaacattctcggcca | |
| | caagctggaatacaactata | |
| | actcccacaatgtgtacatc | |
| | atggccgacaagcaaaagaa | |
| | tggcatcaaggtcaacttca | |
| | agatcagacacaacattgag | |
| | gatggatccgtgcagctggc | |
| | cgaccattatcaacagaaca | |
| | ctccaatcggcgacggccct | |
| | gtgctcctcccagacaacca | |
| | ttacctgtccacccagtctg | |
| | ccctgtctaaagatcccaac | |
| | gaaaagagagaccacatggt | |
| | cctgctggagtttgtgaccg | |
| | ctgctgggatcacacatggc | |
| | atggacgagctgtacaagtg | |
| | agcggccgcggggatccaga | |
| | catgataagatacattgatg | |
| | agtttggacaaaccacaact | |
| | agaatgcagtgaaaaaaatg | |
| | ctttatttgtgaaatttgtg | |
| | atgctattgctttatttgta | |
| | accattataagctgcaataa | |
| | acaagttaacaacaacaatt | |
| | gcattcattttatgtttcag | |
| | gttcagggggaggtgtggga | |
| | ggttttttactagtaaagct | |
| | tgtcgactagagctcgctga | |
| | tcagcctcgactgtgccttc | |
| | tagttgccagccatctgttg | |
| | tttgcccctcccccgtgcct | |
| | tccttgaccctggaaggtgc | |
| | cactcccactgtcctttcct | |
| | aataaaatgaggaaattgca | |
| | tcgcattgtctgagtaggtg | |
| | tcattctattctggggggtg | |
| | ggggtgggcaggacagcaag | |
| | ggggaggattgggaagacaa | |
| | tagcaggcatgctggggaga | |
| | gatctaggaacccctagtga | |
| | tggagttggccactccctct | |
| | ctgcgcgctcgctcgctcac | |
| | tgaggccgcccgggcaaagc | |
| | ccgggcgtcgggcgaccttt | |
| | ggtcgcccggcctcagtgag | |
| | cgagcgagcgcgcagagagg | |
| | gagtggccaac | |
| 2 | ttggccactccctctctgcg | ACMSD AAV |
| | cgctcgctcgctcactgagg | vector |
| | ccgggcgaccaaaggtcgcc | |
| | cgacgcccgggctttgcccg | |
| | ggcggcctcagtgagcgagc | |
| | gagcgcgcagagagggagtg | |
| | gccaactccatcactagggg | |
| | ttcctagatctgaattcggt | |
| | gctagcaccctagttattaa | |
| | tagtaatcaattacggggtc | |
| | attagttcatagcccatata | |
| | tggagttccgcgttacataa | |
| | cttacggtaaatggcccgcc | |
| | tggctgaccgcccaacgacc | |
| | cccgcccattgacgtcaata | |
| | atgacgtatgttcccatagt | |
| | aacgccaatagggactttcc | |
| | attgacgtcaatgggtggac | |
| | tatttacggtaaactgccca | |
| | cttggcagtacatcaagtgt | |
| | atcatatgccaagtacgccc | |
| | cctattgacgtcaatgacgg | |
| | taaatggcccgcctggcatt | |
| | atgcccagtacatgacctta | |
| | tgggactttcctacttggca | |
| | gtacatctacgtattagtca | |
| | tcgctattaccatggtcgag | |
| | gtgagccccacgttctgctt | |
| | cactctccccatctcccccc | |
| | cctccccacccccaattttg | |
| | tatttatttattttttaatt | |
| | attttgtgcagcgatggggg | |
| | cggggggggggggggggcgc | |
| | gcgccaggcggggcggggcg | |
| | gggcgaggggcggggcgggg | |
| | cgaggcggagaggtgcggcg | |
| | gcagccaatcagagcggcgc | |
| | gctccgaaagtttcctttt | |
| | atggcgaggcggcggcggcg | |
| | gcggccctataaaaagcgaa | |
| | gcgcgcggcgggcgggagtc | |
| | gctgcgacgctgccttcgcc | |
| | ccgtgccccgctccgccgcc | |
| | gcctcgcgccgcccgccccg | |
| | gctctgactgaccgcgttac | |
| | tcccacaggtgagcgggcgg | |
| | gacggcccttctcctccggg | |
| | ctgtaattagcgcttggttt | |
| | aatgacggcttgtttctttt | |
| | ctgtggctgcgtgaaagcct | |
| | tgaggggctccgggagggcc | |
| | ctttgtgcgggggggagcgg | |
| | ctcggggggtgcgtgcgtgt | |
| | gtgtgtgcgtggggagcgcc | |
| | gcgtgcggcccgcgctgccc | |
| | ggcggctgtgagcgctgcgg | |
| | gcgcggcgcggggctttgtg | |
| | cgctccgcagtgtgcgcgag | |
| | gggagcgcggccgggggcgg | |
| | tgccccgcggtgcggggggg | |
| | gctgcgaggggaacaaaggc | |
| | tgcgtgcggggtgtgtgcgt | |
| | gggggggtgagcagggggtg | |
| | tgggcgcggcggtcgggctg | |
| | taacccccccctgcaccccc | |
| | ctccccgagttgctgagcac | |
| | ggcccggcttcgggtgcggg | |
| | gctccgtacggggcgtggcg | |
| | cggggctcgccgtgccgggc | |
| | ggggggtggcggcaggtggg | |
| | cggtgccgggcggggggggc | |
| | cgcctcgggccggggagggc | |
| | tcgggggaggggcgcggcgg | |
| | cccccggagcgccggcggct | |
| | gtcgaggcgcggcgagccgc | |
| | agccattgccttttatggta | |
| | atcgtgcgagagggcgcagg | |
| | gacttcctttgtcccaaatc | |
| | tgtgcggagccgaaatctgg | |
| | gaggcgccgccgcaccccct | |
| | ctagcgggcgcggggcgaag | |
| | cggtgcggcgccggcaggaa | |
| | ggaaatgggcggggagggcc | |
| | ttcgtgcgtcgccgcgccgc | |
| | cgtccccttctccctctcca | |
| | gcctcggggctgtccgcggg | |
| | gggacggctgccttcgggg | |
| | gggacggggcagggcggggt | |
| | tcggcttctggcgtgtgacc | |
| | ggcggctctagagcctctgc | |
| | taaccatgttcatgccttct | |
| | tctttttcctacaATGAAAA | |
| | TTGACATCCATAGTCATATT | |
| | CTACCAAAAGAATGGCCAGA | |
| | TCTAAAAAAGAGGTTTGGCT | |
| | ACGGAGGCTGGGTGCAGCTC | |
| | CAACACCACAGCAAGGGAGA | |
| | AGCAAAGTTGTTGAAAGATG | |
| | GGAAAGTCTTCAGAGTGGTG | |
| | CGAGAGAATTGCTGGGATCC | |
| | AGAAGTTCGTATTAGAGAAA | |
| | TGGACCAAAAAGGAGTAACA | |
| | GTGCAAGCCCTTTCCACAGT | |
| | TCCTGTCATGTTTAGCTACT | |
| | GGGCCAAACCTGAGGACACT | |
| | TTAAACCTGTGCCAGCTTTT | |
| | AAACAACGACCTTGCCAGCA | |
| | CCGTTGTGAGCTACCCCAGG | |
| | AGGTTCGTGGGTCTGGGGAC | |
| | GTTGCCCATGCAGGCCCCTG | |
| | AGCTGGCGGTCAAGGAGATG | |
| | GAGCGCTGTGTGAAAGAGCT | |
| | GGGCTTTCCCGGGGTCCAAA | |
| | TTGGCACCCACGTCAACGAG | |
| | TGGGACCTGAACGCGCAGGA | |
| | GCTCTTTCCTGTCTATGCGG | |
| | CAGCCGAAAGGCTGAAGTGT | |
| | TCCCTGTTCGTGCATCCCTG | |
| | GGACATGCAGATGGATGGAC | |
| | GAATGGCCAAATACTGGCTC | |
| | CCTTGGCTTGTAGGAATGCC | |
| | AGCAGAGACCACCATAGCCA | |
| | TTTGCTCCATGATCATGGGT | |
| | GGAGTATTTGAGAAGTTTCC | |
| | CAAACTGAAAGTGTGTTTCG | |
| | CACATGGTGGTGGTGCCTTC | |
| | CCCTTCACAGTGGGAAGAAT | |
| | CTCCCATGGATTCAGCATGC | |
| | GCCCAGATCTGTGTGCCCAG | |
| | GACAACCCCATGAACCCGAA | |
| | GAAATACCTTGGTTCCTTTT | |
| | ACACAGATGCTTTGGTTCAT | |
| | GATCCTCTGTCCCTCAAGCT | |
| | GTTAACAGATGTCATAGGAA | |
| | AGGATAAAGTCATTTTGGGA | |
| | ACCGATTACCCCTTTCCACT | |
| | AGGTGAGCTGGAACCTGGGA | |
| | AACTAATAGAGTCCAT | |
| | GGAAGAATTTGATGAAGAAA | |
| | CAAAGAATAAACTCAAAGCC | |
| | GGCAATGCCCTGGCATTTTT | |
| | GGGTCTTGAGAGAAAACAAT | |
| | TTGAATGAgcggccgcgggg | |
| | atccagacatgataagatac | |
| | attgatgagtttggacaaac | |
| | cacaactagaatgcagtgaa | |
| | aaaaatgctttatttgtgaa | |
| | atttgtgatgctattgcttt | |
| | atttgtaaccattataagct | |
| | gcaataaacaagttaacaac | |
| | aacaattgcattcattttat | |
| | gtttcaggttcagggggagg | |
| | tgtgggaggttttaacccct | |
| | agtgatggagttggccactc | |
| | cctctctgcgcgctcgctcg | |
| | ctcactgaggccgcccgggc | |
| | aaagcccgggcgtcgggcga | |
| | cctttggtcgcccggcctca | |
| | gtgagcgagcgagcgcgcag | |
| | agagggagtggccaac | |
| 3 | aacccctagtgatggagttg | AAV Terminal |
| | gccactccctctctgcgcgc | repeat |
| | tcgctcgctcactgaggccg | |
| | cccgggcaaagcccgggcgt | |
| | cgggcgacctttggtcgccc | |
| | ggcctcagtgagcgagcgag | |
| | cgcgcagagagg | |
| 4 | ATGAAAATTGACATCCATAG | Human ACMSD |
| | TCATATTCTACCAAAAGAAT | CDNA sequence |
| | GGCCAGATCTAAAAAAGAGG | |
| | TTTGGCTACGGAGGCTGGGT | |
| | GCAGCTCCAACACCACAGCA | |
| | AGGGAGAAGCAAAGTTGTTG | |
| | AAAGATGGGAAAGTCTTCAG | |
| | AGTGGTGCGAGAGAATTGCT | |
| | GGGATCCAGAAGTTCGTATT | |
| | AGAGAAATGGACCAAAAAGG | |
| | AGTAACAGTGCAAGCCCTTT | |
| | CCACAGTTCCTGTCATGTTT | |
| | AGCTACTGGGCCAAACCTGA | |
| | GGACACTTTAAACCTGTGCC | |
| | AGCTTTTAAACAACGACCTT | |
| | GCCAGCACCGTTGTGAGCTA | |
| | CCCCAGGAGGTTCGTGGGTC | |
| | TGGGGACGTTGCCCATGCAG | |
| | GCCCCTGAGCTGGCGGTCAA | |
| | GGAGATGGAGCGCTGTGTGA | |
| | AAGAGCTGGGCTTTCCCGGG | |
| | GTCCAAATTGGCACCCACGT | |
| | CAACGAGTGGGACCTGAACG | |
| | CGCAGGAGCTCTTTCCTGTC | |
| | TATGCGGCAGCCGAAAGGCT | |
| | GAAGTGTTCCCTGTTCGTGC | |
| | ATCCCTGGGACATGCAGATG | |
| | GATGGACGAATGGCCAAATA | |
| | CTGGCTCCCTTGGCTTGTAG | |
| | GAATGCCAGCAGAGACCACC | |
| | ATAGCCATTTGCTCCATGAT | |
| | CATGGGTGGAGTATTTGAGA | |
| | AGTTTCCCAAACTGAAAGTG | |
| | TGTTTCGCACATGGTGGTGG | |
| | TGCCTTCCCCTTCACAGTGG | |
| | GAAGAATCTCCCATGGATTC | |
| | AGCATGCGCCCAGATCTGTG | |
| | TGCCCAGGACAACCCCATGA | |
| | ACCCGAAGAAATACCTTGGT | |
| | TCCTTTTACACAGATGCTTT | |
| | GGTTCATGATCCTCTGTCCC | |
| | TCAAGCTGTTAACAGATGTC | |
| | ATAGGAAAGGATAAAGTCAT | |
| | TTTGGGAACCGATTACCCCT | |
| | TTCCACTAGGTGAGCTGGAA | |
| | CCTGGGAAACTAATAGAGTC | |
| | CATGGAAGAATTTGATGAAG | |
| | AAACAAAGAATAAACTCAAA | |
| | GCCGGCAATGCCCTGGCATT | |
| | TTTGGGTCTTGAGAGAAAAC | |
| | AATTTGAATGA | |
| 5 | atgggaaagtcttcagagtg | Human ACMSD |
| | gtgcgagagaattgctggga | coding sequence |
| | tccagaagttcgtattagag | |
| | aaatggaccaaaaaggccaa | |
| | acctgaggacactttaaacc | |
| | tgtgccagcttttaaacaac | |
| | gaccttgccagcaccgttgt | |
| | gagctaccccaggaggttcg | |
| | tgggtctggggacgttgccc | |
| | atgcaggcccctgagctggc | |
| | ggtcaaggagatggagcgct | |
| | gtgtgaaagagctgggcttt | |
| | cccggggtccaaattggcac | |
| | ccacgtcaacgagtgggacc | |
| | tgaacgcgcaggagctcttt | |
| | cctgtctatgcggcagccga | |
| | aaggctgaagtgttccctgt | |
| | tcgtgcatccctgggacatg | |
| | cagatggatggacgaatggc | |
| | caaatactggctcccttggc | |
| | ttgtaggaatgccagcagag | |
| | accaccatagccatttgctc | |
| | catgatcatgggtggagtat | |
| | ttgagaagtttcccaaactg | |
| | aaagtgtgtttcgcacatgg | |
| | tggtggtgccttccccttca | |
| | cagtgggaagaatctcccat | |
| | ggattcagcatgcgcccaga | |
| | tctgtgtgcccaggacaacc | |
| | ccatgaacccgaagaaatac | |
| | cttggttccttttacacaga | |
| | tgctttggttcatgatcctc | |
| | tgtccctcaagctgttaaca | |
| | gatgtcataggaaaggataa | |
| | agtcattttgggaaccgatt | |
| | acccctttccactaggtgag | |
| | ctggaacctgggaaactaat | |
| | agagtccatggaagaatttg | |
| | atgaagaaacaaagaataaa | |
| | ctcaaagccggcaatgccct | |
| | ggcatttttgggtcttgaga | |
| | gaaaacaatttgaa | |
| 6 | mgkssewceriagiqkfvle | Human ACMSD |
| | kwtkkakpedtlnlcqlinn | amino acid |
| | dlastvvsyprrfvglgtlp | sequence |
| | mqapelavkemercvkelgf | |
| | pgvqigthvnewdlnagelf | |
| | pvyaaaerlkcslfvhpwdm | |
| | qmdgrmakywlpwlvgmpae | |
| | ttiaicsmimggvfekfpkl | |
| | kvcfahgggafpftvgrish | |
| | gfsmrpdlcaqdnpmnpkky | |
| | lgsfytdalvhdplslkllt | |
| | dvigkdkvilgtdypfplge | |
| | lepgkliesmeefdeetknk | |
| | lkagnalaflglerkqfe | |
| 7 | tagatctgaattcggtgcta | CBA/CMV |
| | gcaccctagttattaatagt | promoter hybrid |
| | aatcaattacggggtcatta | |
| | gttcatagcccatatatgga | |
| | gttccgcgttacataactta | |
| | cggtaaatggcccgcctggc | |
| | tgaccgcccaacgacccccg | |
| | cccattgacgtcaataatga | |
| | cgtatgttcccatagtaacg | |
| | ccaatagggactttccattg | |
| | acgtcaatgggtggactatt | |
| | tacggtaaactgcccacttg | |
| | gcagtacatcaagtgtatca | |
| | tatgccaagtacgcccccta | |
| | ttgacgtcaatgacggtaaa | |
| | tggccc | |
| | gcctggcattatgcccagta | |
| | catgaccttatgggactttc | |
| | ctacttggcagtacatctac | |
| | gtattagtcatcgctattac | |
| | catggtcgaggtgagcccca | |
| | cgttctgcttcactctcccc | |
| | atctcccccccctccccacc | |
| | cccaattttgtatttattta | |
| | ttttttaattattttgtgca | |
| | gcgatgggggcggggggggg | |
| | gggggggcgcgcgccaggcg | |
| | gggcggggcggggcgagggg | |
| | cggggcggggcgaggcggag | |
| | aggtgcggcggcagccaatc | |
| | agagcggcgcgctccgaaag | |
| | tttccttttatggcgaggcg | |
| | gcggcggcggcggccctata | |
| | aaaagcgaagcgcgcggcgg | |
| | gcg | |
| 8 | tagatctgaattcggtgcta | ACMSD |
| | gcaccctagttattaatagt | expression |
| | aatcaattacggggtcatta | construct |
| | gttcatagcccatatatgga | |
| | gttccgcgttacataactta | |
| | cggtaaatggcccgcctggc | |
| | tgaccgcccaacgacccccg | |
| | cccattgacgtcaataatga | |
| | cgtatgttcccatagtaacg | |
| | ccaatagggactttccattg | |
| | acgtcaatgggtggactatt | |
| | tacggtaaactgcccacttg | |
| | gcagtacatcaagtgtatca | |
| | tatgccaagtacgcccccta | |
| | ttgacgtcaatgacggtaaa | |
| | tggcccgcctggcattatgc | |
| | ccagtacatgaccttatggg | |
| | actttcctacttggcagtac | |
| | atctacgtattagtcatcgc | |
| | tattaccatggtcgaggtga | |
| | gccccacgttctgcttcact | |
| | ctccccatctcccccccctc | |
| | cccacccccaattttgtatt | |
| | tatttattttttaattattt | |
| | tgtgcagcgatgggggcggg | |
| | gggggggggggggcgcgcgc | |
| | caggcggggcgggcgggggc | |
| | gaggggcggggcggggcgag | |
| | gcggagaggtgcggcggcag | |
| | ccaatcagagcggcgcgctc | |
| | cgaaagtttccttttatggc | |
| | gaggcggcggcggcggcggc | |
| | cctataaaaagcgaagcgcg | |
| | cggcgggcgggagtcgctgc | |
| | gacgctgccttcgccccgtg | |
| | ccccgctccgccgccgcctc | |
| | gcgccgcccgccccggctct | |
| | gactgaccgcgttactccca | |
| | caggtgagcgggcgggacgg | |
| | cccttctcctccgggctgta | |
| | attagcgcttggtttaatga | |
| | cggcttgtttcttttctgtg | |
| | gctgcgtgaaagccttgagg | |
| | ggctccgggagggccctttg | |
| | tgcgggggggagcggctcgg | |
| | ggggtgcgtgcgtgtgtgtg | |
| | tgcgtggggagcgccgcgtg | |
| | cggcccgcgctgcccggcgg | |
| | ctgtgagcgctgcgggcgcg | |
| | gcgcggggctttgtgcgctc | |
| | cgcagtgtgcgcgaggggag | |
| | cgcggccgggggcggtgccc | |
| | cgcggtgcggggggggctgc | |
| | gaggggaacaaaggctgcgt | |
| | gcggggtgtgtgcgtggggg | |
| | ggtgagcagggggtgtgggc | |
| | gcggcggtcgggctgtaacc | |
| | cccccctgcacccccctccc | |
| | cgagttgctgagcacggccc | |
| | ggcttcgggtgcggggctcc | |
| | gtacggggcgtggcgcgggg | |
| | ctcgccgtgccgggcggggg | |
| | gtggcggcaggtgggggtgc | |
| | cgggcggggcggggccgcct | |
| | cgggccggggagggctcggg | |
| | ggaggggcgcggcggccccc | |
| | ggagcgccggcggctgtcga | |
| | ggcgcggcgagccgcagcca | |
| | ttgccttttatggtaatcgt | |
| | gcgagagggcgcagggactt | |
| | cctttgtcccaaatctgtgc | |
| | ggagccgaaatctgggaggc | |
| | gccgccgcaccccctctagc | |
| | gggcgcggggcgaagcggtg | |
| | cggcgccggcaggaaggaaa | |
| | tgggcggggagggccttcgt | |
| | gcgtcgccgcgccgccgtcc | |
| | ccttctccctctccagcctc | |
| | ggggctgtccgcggggggac | |
| | ggctgccttcgggggggacg | |
| | gggcagggcggggttcggct | |
| | tctggcgtgtgaccggcggc | |
| | tctagagcctctgctaacca | |
| | tgttcatgccttcttctttt | |
| | tcctacaATGAAAATTGACA | |
| | TCCATAGTCATATTCTACCA | |
| | AAAGAATGGCCAGATCTAAA | |
| | AAAGAGGTTTGGCTACGGAG | |
| | GCTGGGTGCAGCTCCAACAC | |
| | CACAGCAAGGGAGAAGCAAA | |
| | GTTGTTGAAAGATGGGAAAG | |
| | TCTTCAGAGTGGTGCGAGAG | |
| | AATTGCTGGGATCCAGAAGT | |
| | TCGTATTAGAGAAATGGACC | |
| | AAAAAGGAGTAACAGTGCAA | |
| | GCCCTTTCCACAGTTCCTGT | |
| | CATGTTTAGCTACTGGGCCA | |
| | AACCTGAGGACACTTTAAAC | |
| | CTGTGCCAGCTTTTAAACAA | |
| | CGACCTTGCCAGCACCGTTG | |
| | TGAGCTACCCCAGGAGGTTC | |
| | GTGGGTCTGGGGACGTTGCC | |
| | CATGCAGGCCCCTGAGCTGG | |
| | CGGTCAAGGAGATGGAGCGC | |
| | TGTGTGAAAGAGCTGGGCTT | |
| | TCCCGGGGTCCAAATTGGCA | |
| | CCCACGTCAACGAGTGGGAC | |
| | CTGAACGCGCAGGAGCTCTT | |
| | TCCTGTCTATGCGGCAGCCG | |
| | AAAGGCTGAAGTGTTCCCTG | |
| | TTCGTGCATCCCTGGGACAT | |
| | GCAGATGGATGGACGAATGG | |
| | CCAAATACTGGCTCCCTTGG | |
| | CTTGTAGGAATGCCAGCAGA | |
| | GACCACCATAGCCATTTGCT | |
| | CCATGATCATGGGTGGAGTA | |
| | TTTGAGAAGTTTCCCAAACT | |
| | GAAAGTGTGTTTCGCACATG | |
| | GTGGTGGTGCCTTCCCCTTC | |
| | ACAGTGGGAAGAATCTCCCA | |
| | TGGATTCAGCATGCGCCCAG | |
| | ATCTGTGTGCCCAGGACAAC | |
| | CCCATGAACCCGAAGAAATA | |
| | CCTTGGTTCCTTTTACACAG | |
| | ATGCTTTGGTTCATGATCCT | |
| | CTGTCCCTCAAGCTGTTAAC | |
| | AGATGTCATAGGAAAGGATA | |
| | AAGTCATTTTGGGAACCGAT | |
| | TACCCCTTTCCACTAGGTGA | |
| | GCTGGAACCTGGGAAACTAA | |
| | TAGAGTCCATGGAAGAATTT | |
| | GATGAAGAAACAAAGAATAA | |
| | ACTCAAAGCCGGCAATGCCC | |
| | TGGCATTTTTGGGTCTTGAG | |
| | AGAAAACAATTTGAATGAgc | |
| | ggccgcggggatccagacat | |
| | gataagatacattgatgagt | |
| | ttggacaaaccacaactaga | |
| | atgcagtgaaaaaaatgctt | |
| | tatttgtgaaatttgtgatg | |
| | ctattgctttatttgtaacc | |
| | attataagctgcaataaaca | |
| | agttaacaacaacaattgca | |
| | ttcattttatgtttcaggtt | |
| | cagggggaggtgtgggaggt | |
| | ttt | |
| 9 | tagatctgaattcggtgcta | CMV enhancer |
| | gcaccctagttattaatagt | |
| | aatcaattacggggtcatta | |
| | gttcatagcccatatatgga | |
| | gttccgcgttacataactta | |
| | cggtaaatggcccgcctggc | |
| | tgaccgcccaacgacccccg | |
| | cccattgacgtcaataatga | |
| | cgtatgttcccatagtaacg | |
| | ccaatagggactttccattg | |
| | acgtcaatgggtggactatt | |
| | tacggtaaactgcccacttg | |
| | gcagtacatcaagtgtatca | |
| | tatgccaagtacgcccccta | |
| | ttgacgtcaatgacggtaaa | |
| | tggcccgcctggcattatgc | |
| | ccagtacatgaccttatggg | |
| | actttcctacttggcagtac | |
| | atctacgtattagtcatcgc | |
| | tattac | |
| 10 | catggtcgaggtgagcccca | CBA promoter |
| | cgttctgcttcactctcccc | |
| | atctcccccccctccccacc | |
| | cccaattttgtatttattta | |
| | ttttttaattattttgtgca | |
| | gcgatgggggcggggggggg | |
| | gggggggcgcgcgccaggcg | |
| | gggcggggcggggcgagggg | |
| | cggggcggggcgaggcggag | |
| | aggtgcggcggcagccaatc | |
| | agagcggcgcgctccgaaag | |
| | tttccttttatggcgaggcg | |
| | gcggcggcggcggccctata | |
| | aaaagcgaagcgcgcggcgg | |
| | gcg | |
| 11 | ggagtcgctgcgacgctgcc | Synthetic exon |
| | ttcgccccgtgccccgctcc | |
| | gccgccgcctcgcgccgccc | |
| | gccccggctctgactgaccg | |
| | cgttactcccacag | |
| 12 | gtgagcgggcgggacggccc | Synthetic |
| | ttctcctccgggctgtaatt | intron |
| | agcgcttggtttaatgacgg | |
| | cttgtttcttttctgtggct | |
| | gcgtgaaagccttgaggggc | |
| | tccgggagggccctttgtgc | |
| | gggggggagcggctcggggg | |
| | gtgcgtgcgtgtgtgtgtgc | |
| | gtggggagcgccgcgtgcgg | |
| | cccgcgctgcccggcggctg | |
| | tgagcgctgcgggcgcggcg | |
| | cggggctttgtgcgctccgc | |
| | agtgtgcgcgaggggagcgc | |
| | ggccgggggcggtgccccgc | |
| | ggtgcggggggggctgcgag | |
| | gggaacaaaggctgcgtgcg | |
| | gggtgtgtgcgtgggggggt | |
| | gagcagggggtgtgggcgcg | |
| | gcggtcgggctgtaaccccc | |
| | ccctgcacccccctccccga | |
| | gttgctgagcacggcccggc | |
| | ttcgggtgcggggctccgta | |
| | cggggcgtggcgcggggctc | |
| | gccgtgccgggcggggggtg | |
| | gcggcaggtgggggtgccgg | |
| | gcggggcggggccgcctcgg | |
| | gccggggagggctcggggga | |
| | ggggcgcggcggcccccgga | |
| | gcgccggcggctgtcgaggc | |
| | gcggcgagccgcagccattg | |
| | ccttttatggtaatcgtgcg | |
| | agagggcgcagggacttcct | |
| | ttgtcccaaatctgtgcgga | |
| | gccgaaatctgggaggcgcc | |
| | gccgcaccccctctagcggg | |
| | cgcggggcgaagcggtgcgg | |
| | cgccggcaggaaggaaatgg | |
| | gcggggagggccttcgtgcg | |
| | tcgccgcgccgccgtcccct | |
| | tctccctctccagcctcggg | |
| | gctgtccgcggggggacggc | |
| | tgccttcgggggggacgggg | |
| | cagggcggggttcggcttct | |
| | ggcgtgtgaccggcggctct | |
| | agagcctctgctaaccatgt | |
| | tcatgccttcttctttttcc | |
| | taca | |
| 13 | gcggccgcggggatccagac | SV40 polyA |
| | atgataagatacattgatga | |
| | gtttggacaaaccacaacta | |
| | gaatgcagtgaaaaaaatgc | |
| | tttatttgtgaaatttgtga | |
| | tgctattgctttatttgtaa | |
| | ccattataagctgcaataaa | |
| | caagttaacaacaacaattg | |
| | cattcattttatgtttcagg | |
| | ttcagggggaggtgtgggag | |
| | gtttt | |
| 14 | gctcctgggcaacgtgctgg | GFP mRNA |
| | ttattgtgctgtctcatcat | |
| | tttggcaaagtattcctcga | |
| | agatctgctagcaggcgcgc | |
| | ggccgccgccaccatgagca | |
| | agggcgaggaactgttcact | |
| | ggcgtggtcccaattctcgt | |
| | ggaactggatggcgatgtga | |
| | atgggcacaaattttctgtc | |
| | agcggagagggtgaaggtga | |
| | tgccacatacggaaagctca | |
| | ccctgaaattcatctgcacc | |
| | actggaaagctccctgtgcc | |
| | atggccaacactggtcacta | |
| | ccctgacctatggcgtgcag | |
| | tgcttttccagatacccaga | |
| | ccatatgaagcagcatgact | |
| | ttttcaagagcgccatgccc | |
| | gagggctatgtgcaggagag | |
| | aaccatctttttcaaagatg | |
| | acgggaactacaagacccgc | |
| | gctgaagtcaagttcgaagg | |
| | tgacaccctggtgaatagaa | |
| | tcgagctgaagggcattgac | |
| | tttaaggaggatggaaacat | |
| | tctcggccacaagctggaat | |
| | acaactataactcccacaat | |
| | gtgtacatcatggccgacaa | |
| | gcaaaagaatggcatcaagg | |
| | tcaacttcaagatcagacac | |
| | aacattgaggatggatccgt | |
| | gcagctggccgaccattatc | |
| | aacagaacactccaatcggc | |
| | gacggccctgtgctcctccc | |
| | agacaaccattacctgtcca | |
| | cccagtctgccctgtctaaa | |
| | gatcccaacgaaaagagaga | |
| | ccacatggtcctgctggagt | |
| | ttgtgaccgctgctgggatc | |
| | acacatggcatggacgagct | |
| | gtacaagtga | |
| 15 | ttactagtaaagcttgtcga | ACMSD shRNA |
| | ctagagctcgctgatcagcc | |
| | tcgactgtgccttctagttg | |
| | ccagccatctgttgtttgcc | |
| | cctcccccgtgccttccttg | |
| | accctggaaggtgccactcc | |
| | cactgtcctttcctaataaa | |
| | atgaggaaattgcatcgcat | |
| | tgtctgagtaggtgtcattc | |
| | tattctggggggtggggtgg | |
| | ggcaggacagcaagggggag | |
| | gattgggaagacaatagcag | |
| | gcatgctggggagagatcta | |
| | gg | |
| 16 | AATTCATATTTGCATGTCGC | H1 promoter |
| | TATGTGTTCTGGGAAATCAC | |
| | CATAAACGTGAAATGTCTTT | |
| | GGATTTGGGAATCTTATAAG | |
| | TTCTGTATGAGACCACTCGG | |
| | ATCCG | |

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Expression of ACMSD in Neuronal Cells Improves Motor Symptoms in a Huntington's Disease Model

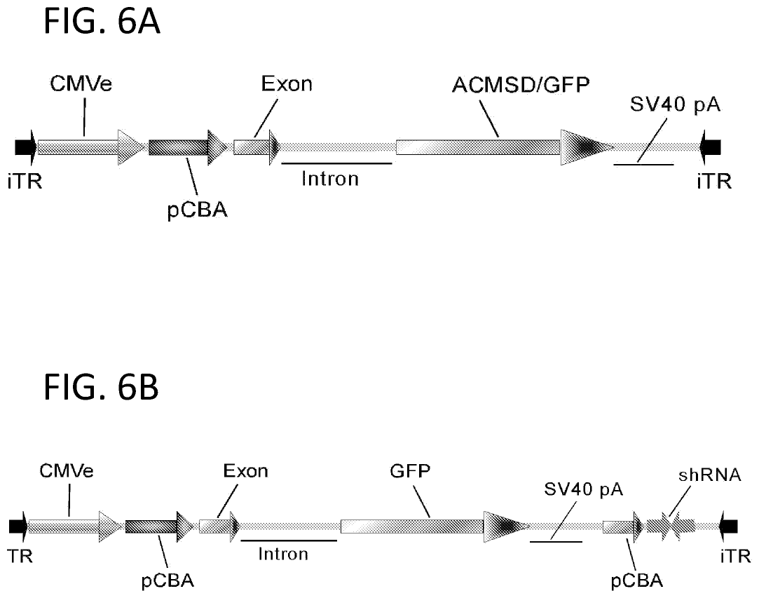

Huntington's disease model (YAC 128) mice were treated intraparenchymally with a rAAV vector expressing ACMSD (FIG. 6A), or a rAAV vector expressing as a negative control (FIG. 6B). The ACMSD (type 1) clone used for over-expression was cloned using human cDNA and inserted into a rAAV backbone behind a hybrid chicken beta-actin/Cytomegalovirus promoter (FIG. 6A). All pseudotyped rAAV 2/5 vectors were produced using a triple transfection protocol followed by iodixanol purification and FPLC fractionation as previously described. In all aims, animals received a 2 μl bilateral injection of either vector (rAAV2/5-GFP as vector control) (˜1.0×10¹³ vector genomes/ml (vg/ml)) stereotaxically delivered using pulled pre-siliconized glass micropipettes. Coordinates are amygdala: AP −2.1 mm, ML +/−4.0 mm, and DV −8.0 mm. PFC: AP +2.7 mm, ML +/−0.5 mm, and DV −4.5 mm. Hippocampus: AP −3.3 mm, ML +/−2.6 mm, and DV −2.6 mm.

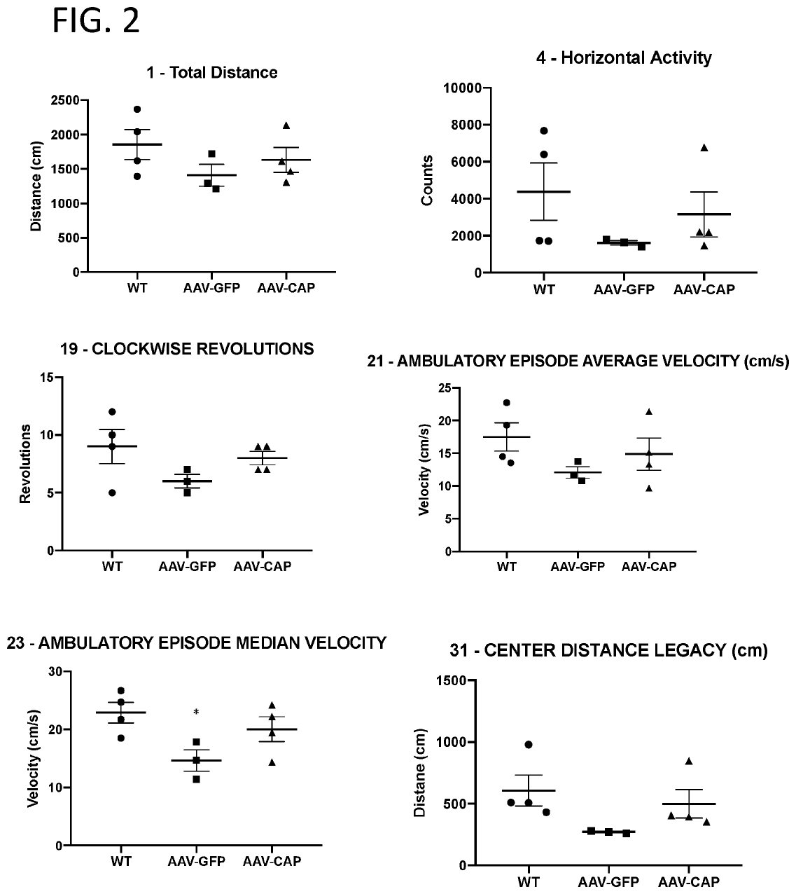

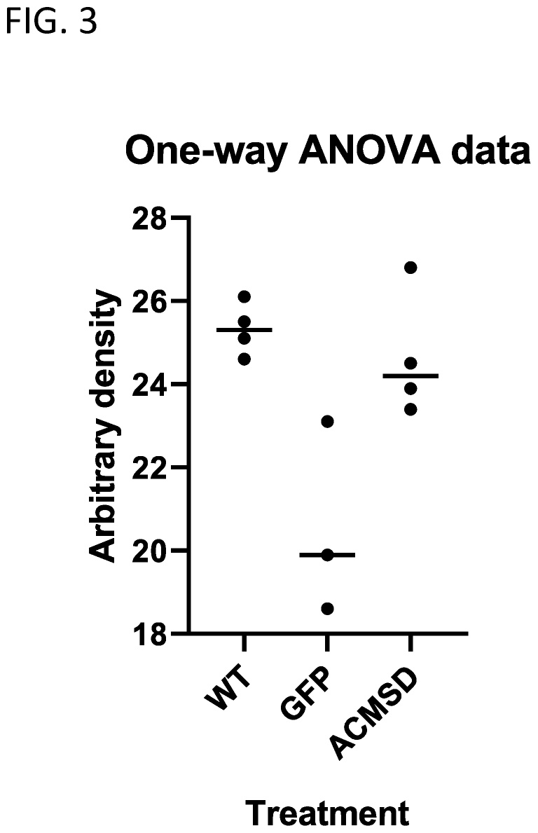

Catwalk analyses (FIG. 2) show improved behavior in ACMSD treated animals. Moreover, histological analysis of striatal atrophy (FIG. 3. Raw data in Table 1) show that ACMSD overexpression results in protection of striatal neurons. The results show that expression of ACMSD in the HD mouse model provides a therapeutic benefit for ACMSD.

TABLE 1
Striatal atrophy is prevented

| | |
|---|---|
| Number of families | 1 |
| Number of | 3 |
| comparisons per | |
| family | |
| Alpha | 0.5 |

| Tukey's multiple | | 95.00% CI | |
|---|---|---|---|
| comparisons test | Mean Diff. | of Diff. | Significant? |
| WT vs GFP | 4.792 | 1.455 to 8.192 | Yes |
| WT vs ACMSD | 0.6750 | −2.414 to 3.764 | No |
| GFP vs ACMSD | −4.117 | −7.454 to −0.7796 | Yes |

| Test details | Mean 1 | Mean 2 | Mean Diff. |
|---|---|---|---|
| WT vs GFP | 25.33 | 20.53 | 4.792 |
| WT vs ACMSD | 25.33 | 24.65 | 0.6750 |
| GFP vs ACMSD | 20.53 | 24.65 | −4.117 |

In some aspects, an AAV vector comprising an expression construct expressing an shRNA targeting a nucleic acid sequence in a nucleic acid sequence encoding ACMSD. Small interfering RNA (siRNA) sequences were designed from the published rat ACMSD sequence and the control siRNA comprised a scrambled nucleotide sequence. Duplexed shRNA encoding nucleotides is inserted into a rAAV2 backbone (expressed from the H1 promoter). ACMSD knockdown was confirmed in vitro by co-transfection with the target cDNA in 293 cells. In addition, the vector backbone contains a transduction marker in the form of green fluorescent protein (GFP) (FIG. 6B).

### Example 2. Deletion of ACMSD Creates a Parkinson's Disease Phenotype

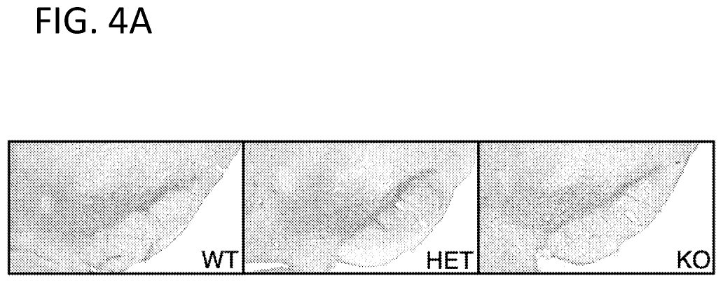

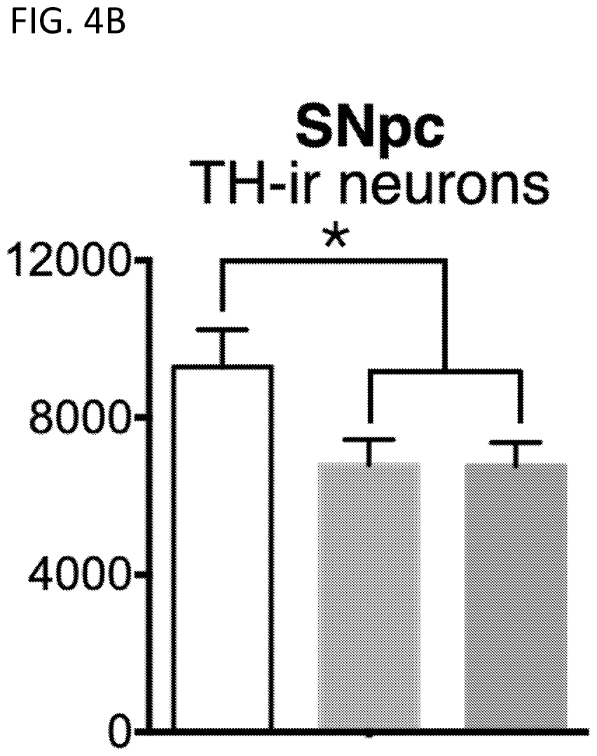

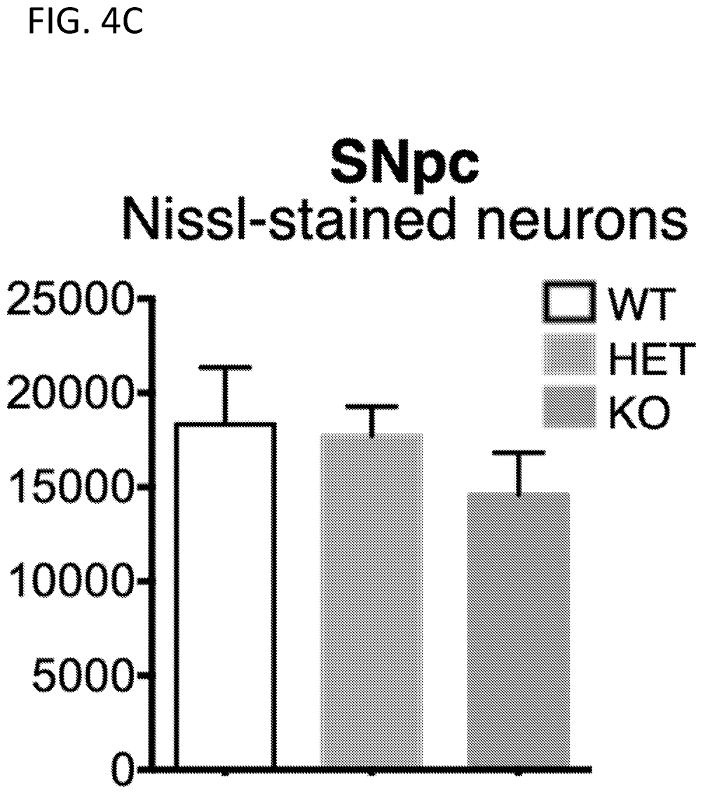

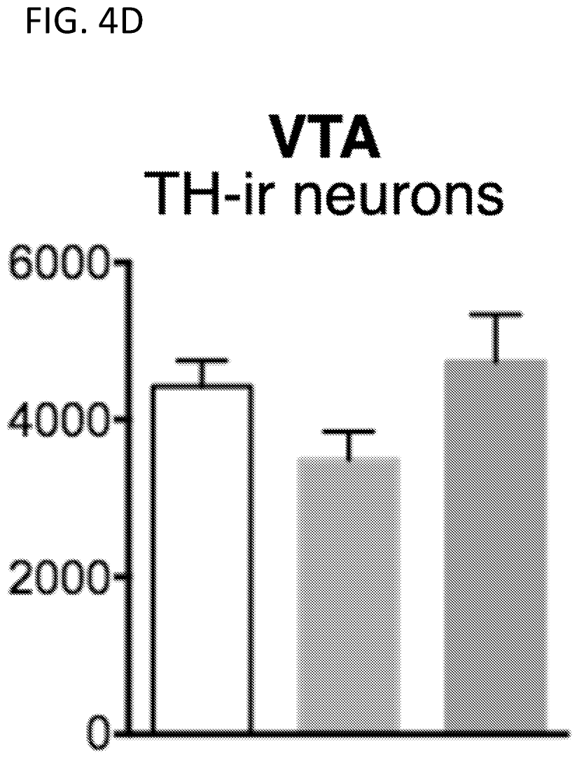

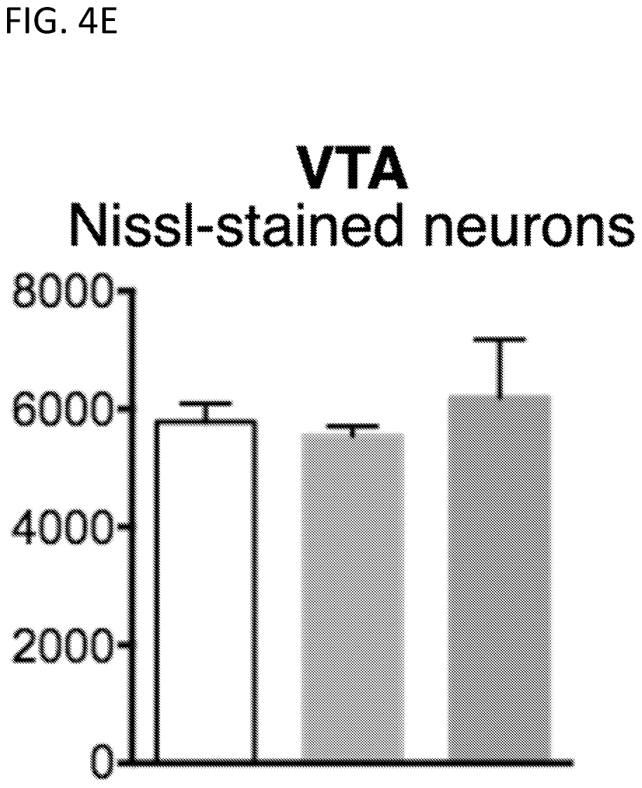

ACMSD was knocked down in the brain of a WT mouse. Animals were sacrificed at 6 months age and histological analysis as shown in FIG. 4A show that removal of ACMSD is detrimental to midbrain dopamine neurons. Quantification of tyrosine hydroxylase (TH)-containing neurons (TH-immunoreactive; TH-ir neurons) shows that these neurons are significantly decreased in SNpc of ACMSD KO mice (FIG. 4B). Similarly, FIG. 4C shows a trend towards overall reduction of SNpc neurons due to removal of ACMSD. This confirms that TH neurons are lost, and not merely the loss of the TH phenotype in Nissl-stained neurons. Conversely, there was no change in TH-ir or Nissl-stained neurons in the ventral tegmental area (VTA) in ACMSD knockout brains (FIG. 4D, FIG. 4E).

Results show that deletion of ACMSD is toxic to the same dopamine neurons that die in PD. The animals exhibit a PD phenotype.

### Example 3. Expression of ACMSD in Neuronal Cells Provides Neuroprotection in a Parkinson's Disease Model

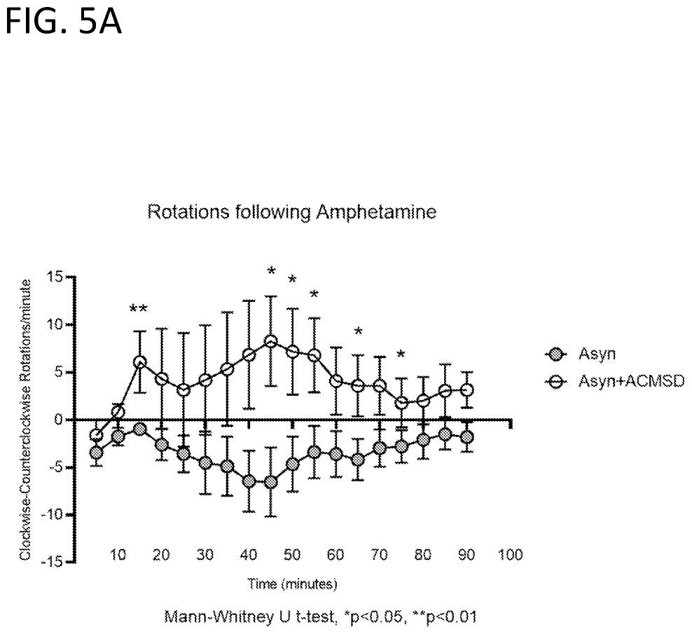

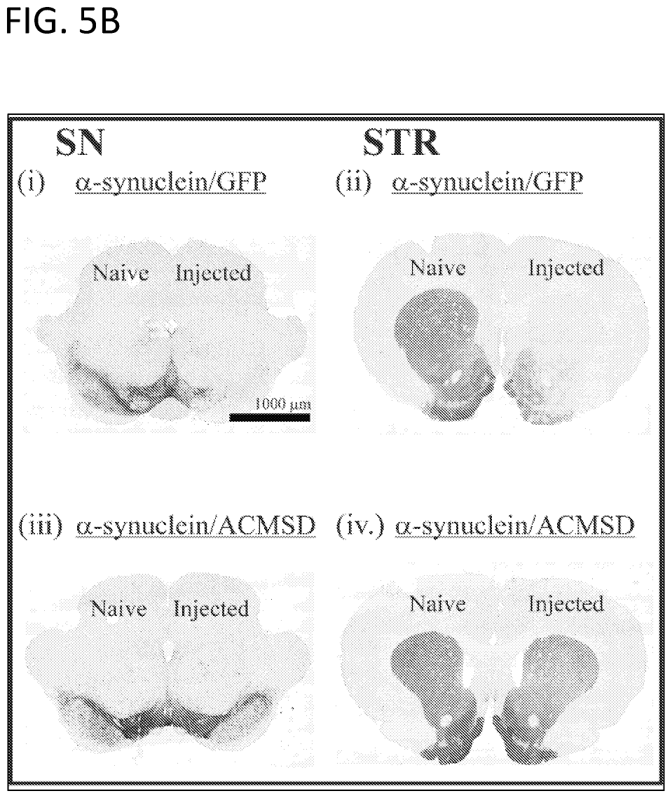

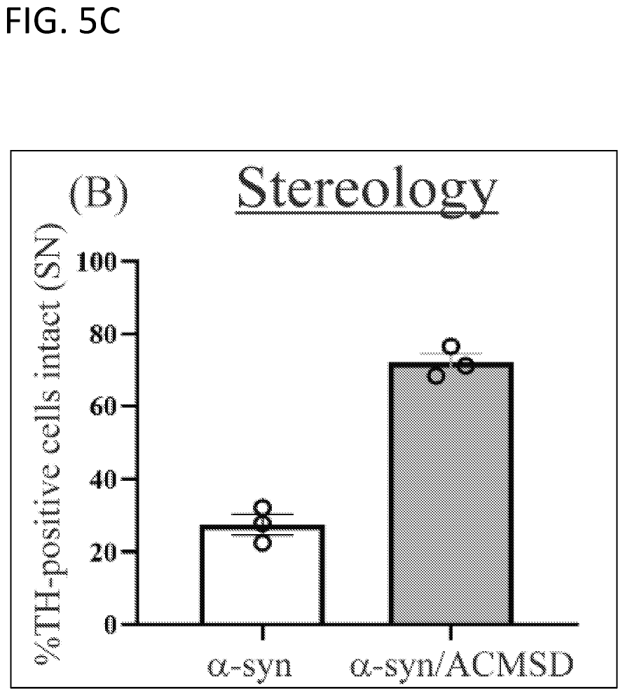

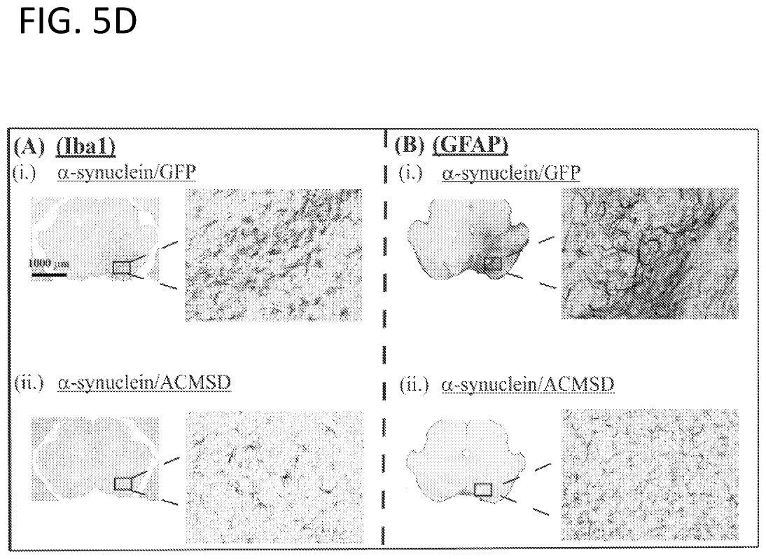

An α-synuclein (Asyn) Parkinson's disease rat model was used to assess the effects of ACMSD expression in neuronal cells. Asyn mice were injected intraparenchymally with a rAAV vector expressing ACMSD, or a rAAV vector expressing GFP as a negative control. The amphetamine (AMPH)-induced rotation test was used to monitor the effect of expression of ACMSD in neuronal cells (FIG. 5A). The results show that expression of ACMSD in the mouse model provides a therapeutic benefit for ACMSD.

High titer α-syn (1*10¹³ vg/ml) was delivered to the right rat substantia nigra together with the rAAV vector expressing FLEX-GFP (control; FIG. 5B top) or the rAAV vector expressing ACMSD (FIG. 5B bottom). Qualitative assessment of TH immunoreactivity 8-weeks following vector delivery shows near complete nigrostriatal denervation in control animals (panels I and ii). ACMSD overexpression was associated with higher TH immunoreactivity both in the SN (panel iii) and terminals (panel iv). Stereological cell count of pilot animals suggest that ACMSD is neuroprotective (FIG. 5C).

Qualitative immunohistochemical assessment from animals receiving AAV-α-syn exhibit gliosis as seen by marked increases in lba1 and GFAP in control animals (FIG. 5D, panels Ai., Bi.), which are absent in animals injected with AAV-αsyn/ACMSD (FIG. 5D, panels Aii., Bii.). The results show that ACMSD overexpression reduces neuroinflammation.

### Example 4. Rescue of Dopamine Neurons in a Mouse Model of Parkinson's Disease

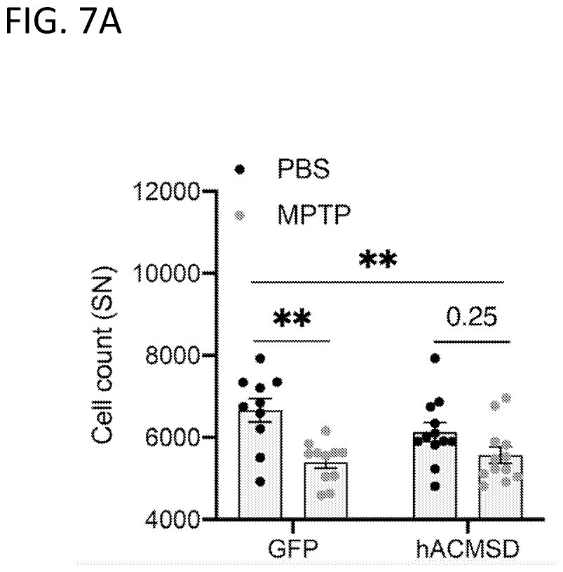

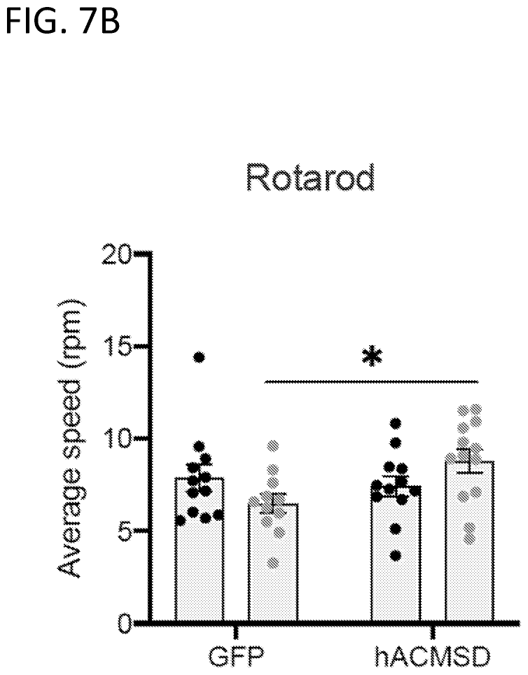

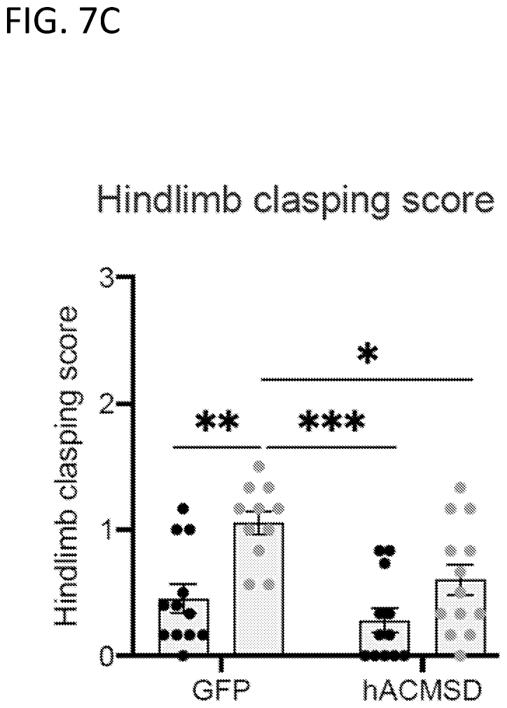

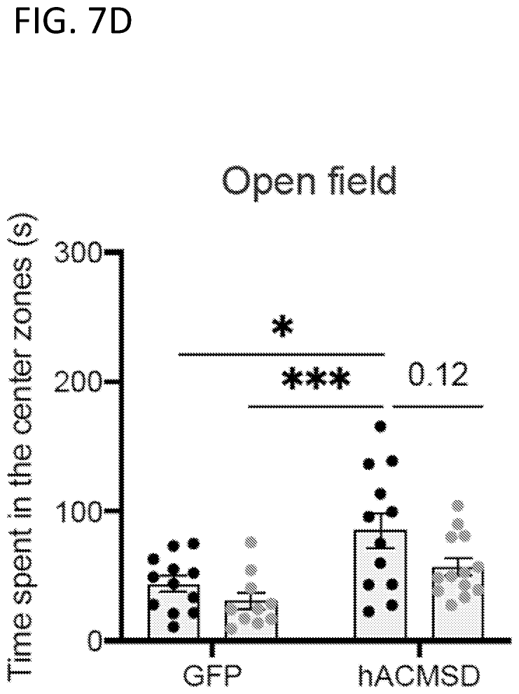

Mice received the neurotoxin MPTP, which is toxic to the dopamine neurons in the substantia nigra. MPTP mice and control animals (treated with PBS were then either treated with the control (GFP) vector or the vector expressing ACMSD to prevent this toxicity. FIG. 7A shows that there is a significant difference in the number of dopamine neurons between MPTP treated and untreated (PBS) animals. However, in the group of mice that were treated with the ACMSD containing vector, there is no effect of the neurotoxin (no significant difference between MPTP treated and PBS treated groups in terms of dopamine neurons). This illustrates that treatment with the vector containing the ACMSD enzyme sequence has a neuroprotective effect and can rescue dopamine neurons from the MPTP toxicity.

Results of behavioral testing of the animals described above are shown in FIGS. 7B-7D. In FIG. 7B, the mice were tested for motor function on a rotarod. Although there is no clear effect of the toxin in this model, the data shows that the mice receiving the ACMSD containing vector are able to remain on the rotarod with a higher speed, and thus appear to have improved motor performance in this setting. In FIG. 7C, there is a clear effect of the toxin on the hindlimb clasping, in that animals that received the MPTP toxin exhibit a higher clasping score. However, in the mice that were treated with the ACMSD containing vector, this clasping behavior is significantly reduced. Thus, the ACMSD vector partially restored motor function after MPTP lesion in this testing setting. In FIG. 7D, it can be seen that mice that received the ACMSD containing vector spend more time in the center of the open field, irrespective of if they had an MPTP lesion or not. Spending more time in the center of an open field is a sign of decreased anxiety. Thus, the results show that mice treated with the vector containing ACMSD exhibit less anxiety, independent of the toxin treatment.

### Example 5. ACMSD Overexpression Reduces Neurodegeneration in the P301S Model of Alzheimer's Disease

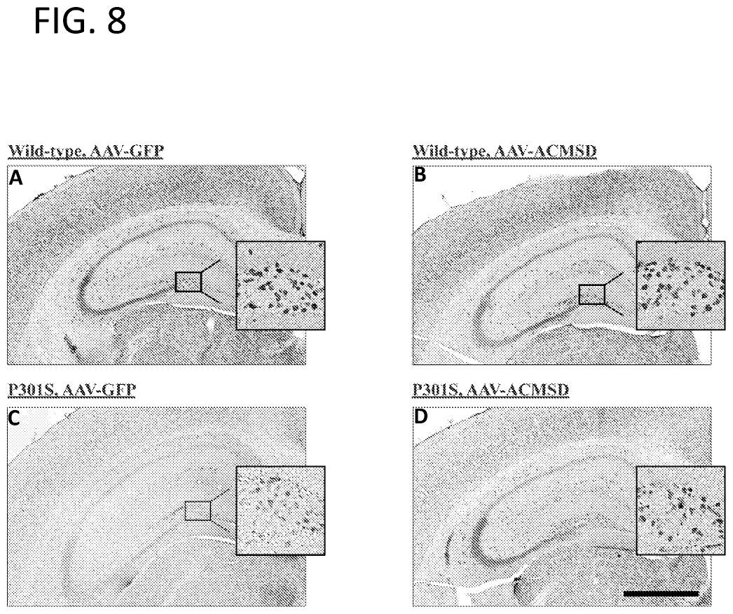

HuC/D (pan neuronal marker) immunoreactivity was assayed in animals receiving hippocampal injections with AAV-GFP (FIG. 8 panel A) or AAV-ACMSD (FIG. 8 panel B). Results show lack of neurotoxicity in wildtype control animals. However, P301S animals receiving hippocampal AAV-GFP show a reduction in the number of neurons (FIG. 8 panel C). This neuronal loss is rescued with hippocampal ACMSD overexpression (FIG. 8 panel D).

### Example 6. ACMSD Overexpression Reduces Neuroinflammation in a Model of Alzheimer's Disease

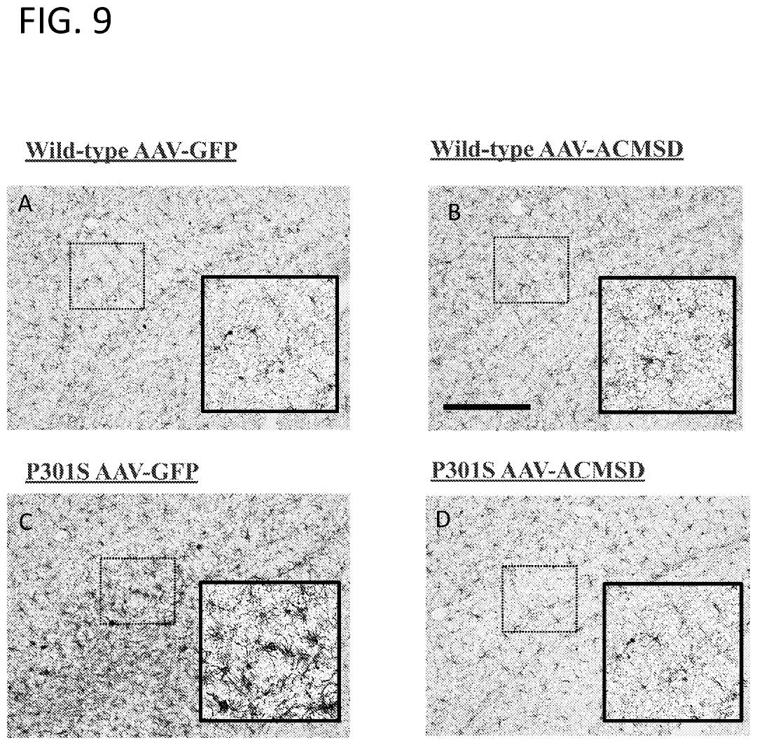

The P301S mouse model of AD exhibits significant inflammation/microgliosis as assessed by lba1 immunoreactivity in the hippocampus (FIG. 9 panel C) compared to wildtype mice (FIG. 9 panels A, B). However, AAV-mediated overexpression of ACMSD reduces this inflammation to levels comparable to wildtype animals (FIG. 9 panel D). No inflammation can be seen in wildtype animals treated with AAV-ACMSD.

### Example 7. Expression of ACMSD in Neuronal Cells Improves ALS Disease Symptoms in an Animal Model

A rAAV vector expressing ACMSD are injected into the brain and spinal cord in an ALS animal model. A rAAV vector expressing GFP is used as a negative control, and results are compared to wild type animals. ALS symptoms in the animals were measured.

What is claimed is:

1. An expression construct for expressing an aminocarboxymuconate semialdehyde decarboxylase (ACMSD) protein in a target cell, the expression construct comprising:
a. a promoter operably linked to a nucleic acid sequence encoding a programmable nucleic acid modification system targeted to a nucleic acid sequence in a nucleotide sequence encoding the ACMSD protein; or
b. a promoter operably linked to a nucleotide sequence encoding the ACMSD protein.

2. The expression construct of claim 1, wherein a cell comprising the expression construct comprises restored deficiency of ACMSD expression in the target cell.

3. The expression construct of claim 1 or 2, wherein a cell comprising the expression construct comprises ectopically expressed ACMSD in the target cell.

4. The expression construct of any one of the preceding claims, wherein a cell comprising the expression construct comprises increased the expression of the ACMSD protein to levels higher than levels normally found in cells.

5. The expression construct of any one of the preceding claims, wherein a cell comprising the expression construct comprises increased the expression of the ACMSD protein to levels higher than levels in cells having defective levels of ACMSD expression.

6. The expression construct of any one of the preceding claims, wherein the amino acid sequence of the ACMSD protein comprises about 75% or more, 85% or more, 95% or more, or 100% sequence identity with SEQ ID NO: 6.

7. The expression construct of any one of the preceding claims, wherein the target cell is liver, kidney, placenta, brain, cancer cells or tumors, cells of the immune system, or any combination thereof.

8. The expression construct of any one of the preceding claims, wherein the target cell is a cell of the central nervous system.

9. The expression construct of any one of the preceding claims, wherein the target cell is a neural cell.

10. The expression construct of any one of the preceding claims, wherein the target cell is a cell of glial lineage.

11. The expression construct of any one of the preceding claims, wherein the target cell is an astrocyte.

12. The expression construct of any one of the preceding claims, wherein the target cell is a glial cell.

13. The expression construct of any one of the preceding claims, wherein the target cell is in a subject suffering from inflammation, or a disorder associated with inflammation.

14. The expression construct of any one of the preceding claims, wherein the target cell is in a subject suffering from a condition associated with inflammation, oxidative stress, protein aggregation, energy failure, toxic exposure such as exposure to pollutants, genetic factors, environmental conditions, immune system activity, or any combination thereof.

15. The expression construct of any one of the preceding claims, wherein the target cell is in a subject suffering from a neurological disorder or a psychiatric disorder.

16. The expression construct of claim 15, wherein the neurological disorder is Parkinson's disease, Alzheimer's disease, ALS, Huntington's disease, brain ischemia, CNS infections and autoimmune disorders, or any combination thereof.

17. The expression construct of claim 15, wherein the psychiatric disorder is a mood disorder, schizophrenia, suicidality, or any combination thereof.

18. The expression construct of any one of the preceding claims, wherein the promoter is a tissue or cell-specific promoter control sequence.

19. The expression construct of claim 18, wherein the tissue or cell-specific promoter control sequence is an interneuron specific promoter, a hippocampal promoter, a glial promoter a dopamine beta-hydroxylase promoter, a glutamatergic neuron promoter, a tyrosine hydroxylase promoter, a motor neuron promoter, a serotonergic promoter, a microglial promoter, an astrocyte specific promoter, an oligodendrocyte specific promoter, or any combination thereof.

20. The expression construct of claim 19, wherein the tissue or cell-specific promoter control sequence is a microglial promoter.

21. The expression construct of claim 19, wherein the tissue or cell-specific promoter control sequence is an astrocyte specific promoter.

22. The expression construct of any one of the preceding claims, wherein the engineered vector-mediated system comprises a nucleic acid expression construct comprising a promoter operably linked to a nucleotide sequence encoding the ACMSD protein.

23. The expression construct of claim 22, wherein the ubiquitous promoter is a CBA/CMV promoter hybrid.

24. The expression construct of claim 23, wherein the nucleic acid sequence of the expression construct comprises about 75% or more, 85% or more, 95% or more, or 100% sequence identity with SEQ ID NO: 8.

25. The expression construct of claim 23, wherein the nucleic acid sequence of the CBA/CMV promoter hybrid comprises about 75% or more, 85% or more, 95% or more, or 100% sequence identity with SEQ ID NO: 7.

26. The expression construct of any of the preceding claims, further comprising a nucleic acid delivery vector comprising the nucleic acid expression construct for delivering the nucleic acid expression construct to the target cell.

27. The expression construct of claim 26, wherein the delivery system is a viral vector.

28. The expression construct of claim 27, wherein the viral vector exhibits tropism to the target cell.

29. The expression construct of claim 27, wherein the viral vector is a recombinant adeno-associated virus (rAAV) vector encapsidating the nucleic acid construct for delivering the construct to the target cell.

30. The expression construct of claim 27, wherein the rAAV vector comprises an AAV2 capsid protein comprising a Y444F, Y500F, Y730F amino acid substitution, or any combination thereof, or corresponding substitutions in the capsid protein of another AAV serotype.

31. The expression construct of claim 27, wherein the rAAV vector comprises an AAV2 capsid protein comprising a R585S, R588T, and R487G amino acid substitution, or any combination thereof, or corresponding substitutions in the capsid protein of another AAV serotype.

32. The expression construct of claim 27, wherein the AAV vector comprises a promoter operably linked to a nucleotide sequence encoding the ACMSD protein, wherein the nucleic acid sequence of the AAV vector comprises about 75% or more, 85% or more, 95% or more, or 100% sequence identity with SEQ ID NO: 2.

33. The expression construct of claim any of claims 1-17, wherein the programmable nucleic acid modification system comprises an interfering nucleic acid molecule having a nucleotide sequence complementary to a nucleic acid sequence within a nucleic acid sequence encoding the ACMSD protein.

34. The expression construct of claim 33, wherein the interfering nucleic acid molecule is selected from an antisense molecule, siRNA molecules, single-stranded siRNA molecules, miRNA molecules, piRNA molecules, lncRNA molecules, and shRNA molecules.

35. The expression construct of claim 34, wherein the interfering nucleic acid molecule is a shRNA.

36. The expression construct of claim 35, wherein the shRNA molecule comprises a nucleotide sequence complementary to a target sequence within a nucleic acid sequence encoding the ACMSD protein.

37. The expression construct of claim 36, wherein the nucleic acid sequence of the shRNA comprises about 75% or more, 85% or more, 95% or more, or 100% sequence identity with SEQ ID NO: 15.

38. An engineered vector-mediated system for expressing an aminocarboxymuconate semialdehyde decarboxylase (ACMSD) protein in a target cell, the system comprising:
a. a nucleic acid expression construct of any one of claims 1-37 comprising:
i. a promoter operably linked to a nucleic acid sequence encoding a programmable nucleic acid modification system targeted to a nucleic acid sequence in a nucleotide sequence encoding the ACMSD protein; or
ii. a promoter operably linked to a nucleotide sequence encoding the ACMSD protein; and
b. a nucleic acid delivery vector comprising the nucleic acid expression construct for delivering the nucleic acid expression construct to the target cell.

39. The engineered vector-mediated system of claim 38, wherein the delivery system is a viral vector.

40. The engineered vector-mediated system of claim 38, wherein the viral vector exhibits tropism to the target cell.

41. The engineered vector-mediated system of claim 38, wherein the viral vector is a recombinant adeno-associated virus (rAAV) vector encapsidating the nucleic acid construct for delivering the construct to the target cell.

42. The engineered vector-mediated system of claim 38, wherein the rAAV vector comprises an AAV2 capsid protein comprising a Y444F, Y500F, Y730F amino acid substitution, or any combination thereof, or corresponding substitutions in the capsid protein of another AAV serotype.

43. The engineered vector-mediated system of claim 38, wherein the rAAV vector comprises an AAV2 capsid protein comprising a R585S, R588T, and R487G amino acid substitution, or any combination thereof, or corresponding substitutions in the capsid protein of another AAV serotype.

44. The engineered vector-mediated system of claim 38, wherein the engineered vector-mediated system comprises a promoter operably linked to a nucleotide sequence encoding the ACMSD protein, wherein the nucleic acid sequence of the AAV vector comprises about 75% or more, 85% or more, 95% or more, or 100% sequence identity with SEQ ID NO: 2.

45. A method of treating a disease condition associated with a pathological condition, the method comprising expressing an ACMSD protein in a target cell in a subject in need thereof by administering to the subject a therapeutically effective amount of the expression construct of any one of claims 1-37 or a vector-mediated system of claims 38-44 for expressing an ACMSD protein in a target cell.

46. The method of claim 45, wherein the method comprises restoring a deficiency of ACMSD expression in the target cell.

47. The method of claim 45, wherein the method comprises ectopically expressing ACMSD in the target cell.

48. The method of claim 45, wherein the method comprises increasing the expression of the ACMSD protein to levels higher than levels normally found in cells having normal or cells having defective levels of ACMSD expression.

49. The method of claim 45, wherein the pathological conditions include abnormal inflammation, oxidative stress, protein aggregation, energy failure, toxic exposure such as exposure to pollutants, genetic factors, environmental conditions, immune system activity, or any combination thereof

50. The method of any one of the preceding claims, wherein the target cell is a cell of the central nervous system.

51. The method of any one of the preceding claims, wherein increasing the expression of ACMSD in cells of the nervous system provides a neurotrophic effect.

52. The method of any one of the preceding claims, wherein the disease condition is associated with quinolinic acid.

53. The method of any one of the preceding claims, wherein the disease condition is associated with inflammation.

54. The method of any one of the preceding claims, wherein the disease condition is associated with neuroinflammation.

55. The method of any one of the preceding claims, wherein the disease condition is a neurological or psychiatric disorder.

56. The method of claim 55, wherein the neurological disorder is Parkinson's disease, Alzheimer's disease, ALS, Huntington's disease, brain ischemia, CNS infections and autoimmune disorders, or any combination thereof.

57. The method of claim 55, wherein the psychiatric disorder is a mood disorder, schizophrenia, suicidality, or any combination thereof.

58. The method of claim 45, wherein the disease condition is Huntington's disease, wherein the engineered vector-mediated system comprises a nucleic acid sequence comprising about 75% or more, 85% or more, 95% or more, or 100% sequence identity with SEQ ID NO: 2, and wherein the method comprises injecting a therapeutically effective amount of the engineered vector-mediated system in the basal ganglia of a subject in need thereof.

59. The method of claim 45, wherein the disease condition is Parkinson's disease, wherein the engineered vector-mediated system comprises a nucleic acid sequence comprising about 75% or more, 85% or more, 95% or more, or 100% sequence identity with SEQ ID NO: 2, and wherein the method comprises injecting a therapeutically effective amount of the engineered vector-mediated system in the substantia nigra of a subject in need thereof.

60. The method of claim 45, wherein the disease condition is Alzheimer's disease, wherein the engineered vector-mediated system comprises a nucleic acid sequence comprising about 75% or more, 85% or more, 95% or more, or 100% sequence identity with SEQ ID NO: 2, and wherein the method comprises injecting a therapeutically effective amount of the engineered vector-mediated system in the hippocampus of a subject in need thereof.

61. A method of providing a general protective or therapeutic effect in a cell or tissue in a subject in need thereof, the method comprising expressing the ACMSD protein in a target cell in the nervous system of the subject by administering to the subject a therapeutically effective amount of the expression construct of any one of claims 1-37 or a vector-mediated system of claims 38-44 for expressing an ACMSD protein in a target cell.

62. The method of claim 61, wherein the method increases ACMSD expression in the cell or tissue of the subject.

63. The method of claim 61 or 62, wherein the method increases the expression of the ACMSD protein to levels higher than levels normally found in cells having normal or cells having defective levels of ACMSD expression.

64. The method of any one of the preceding claims, wherein providing a general protective effect comprises providing protection from pathological conditions associated with inflammation, oxidative stress, protein aggregation, energy failure, toxic exposure such as exposure to pollutants, genetic factors, environmental conditions, immune system activity, or any combination thereof.

65. The method of any one of the preceding claims, wherein providing a general protective effect comprises providing protection from disease conditions associated with quinolinic acid.

66. The method of any one of the preceding claims, wherein providing a general protective effect comprises providing protection from disease conditions associated with inflammation.

67. The method of any one of the preceding claims, wherein the general protective effect is a neurotrophic effect.

68. The method of any one of the preceding claims, wherein providing a general protective effect comprises providing protection from disease conditions associated with neuroinflammation.

69. The method of any one of the preceding claims, wherein providing a general protective effect comprises providing protection from Parkinson's disease, Alzheimer's disease, ALS, Huntington's disease, brain ischemia, CNS infections and autoimmune disorders, mood disorders, schizophrenia, or any combination thereof.

70. The method of claim 69, wherein the disease condition is Huntington's disease, wherein the engineered vector-mediated system comprises a nucleic acid sequence comprising about 75% or more, 85% or more, 95% or more, or 100% sequence identity with SEQ ID NO: 2, and wherein the method comprises injecting a therapeutically effective amount of the engineered vector-mediated system in the basal ganglia of a subject in need thereof.

71. The method of claim 69, wherein the disease condition is Parkinson's disease, wherein the engineered vector-mediated system comprises a nucleic acid sequence comprising about 75% or more, 85% or more, 95% or more, or 100% sequence identity with SEQ ID NO: 2, and wherein the method comprises injecting a therapeutically effective amount of the engineered vector-mediated system in the substantia nigra of a subject in need thereof.

72. The method of claim 69, wherein the disease condition is Alzheimer's disease, wherein the engineered vector-mediated system comprises a nucleic acid sequence comprising about 75% or more, 85% or more, 95% or more, or 100% sequence identity with SEQ ID NO: 2, and wherein the method comprises injecting a therapeutically effective amount of the engineered vector-mediated system in the hippocampus of a subject in need thereof.

73. A method of providing a protective or therapeutic effect against Huntington's disease in a subject in need thereof, the method comprising injecting brain wide, into the caudate/putamen (striatum) of the subject or any combination thereof a therapeutically effective amount of an engineered vector-mediated system of claims 38-44 comprising an interneuron specific promoter, a neuron specific promoter, or a glial promoter.

74. A method of providing a protective or therapeutic effect against Parkinson's disease in a subject in need thereof, the method comprising injecting brain wide, into the caudate/putamen, substantia nigra, cortex, brainstem, amygdala, autonomic nervous system, enteric nervous system of the subject or any combination thereof a therapeutically effective amount of an engineered vector-mediated system of claims 38-44 comprising a neuron specific promoter or a glial promoter.

75. A method of providing a protective or therapeutic effect against Alzheimer's disease in a subject in need thereof, the method comprising injecting brain wide, into the cortex, the hippocampus, the entorhinal cortex of the subject or any combination thereof a therapeutically effective amount of engineered vector-mediated system of claims 38-44.

76. A method of providing a neurotrophic effect to a cell of the nervous system in a subject in need thereof, the method comprising expressing the ACMSD protein in the cell in the subject by administering to the subject a therapeutically effective amount of the expression construct of any one of claims 1-37 or a vector-mediated system of claims 38-44 for expressing an ACMSD protein in a target cell.

77. A method of providing an anti-inflammatory effect to a target neuronal cell in a subject in need thereof, the method comprising expressing the ACMSD protein in the cell by administering to the subject a therapeutically effective amount of the expression construct of any one of claims 1-37 or a vector-mediated system of claims 38-44 for expressing an ACMSD protein in a target cell.

78. A library of engineered vector-mediated expression systems comprising a plurality of the engineered vector mediated systems of any one of claims 38-44 for expressing ACMSD.

79. The library of claim 78, wherein the library comprises a plurality of engineered systems, wherein each of the plurality of systems comprises a non-cell or non-tissue-specific delivery system, and a plurality of cell-or tissue-specific ACMSD expression systems.

80. The library of claim 78, wherein the non-cell or tissue-specific delivery system is a non-cell specific rAAV vector.

81. The library of claim 78, wherein the library comprises a plurality of engineered systems for expressing ACMSD, wherein each of the plurality of systems comprises a plurality of cell or tissue-specific delivery systems and a protein expression system for ubiquitous expression of ACMSD.

82. The library of claim 78, wherein the cell or tissue-specific delivery system is an rAAV vector having tropism to a target cell or tissue.

83. A nucleic acid construct encoding the expression construct of any one of claims 1-37 or a vector-mediated system of claims 38-44 for expressing an ACMSD protein in a target cell.

84. The nucleic acid construct of claim 83, wherein the nucleic acid sequence of the ACMSD expression construct comprises about 75% or more, 85% or more, 95% or more, or 100% sequence identity with SEQ ID NO: 8.

85. A kit comprising at least one expression construct of any one of claims 1-37 or vector-mediated system of claims 38-44 or both for expressing an ACMSD protein in a target cell, or at least one library of engineered systems of any one of claims 78-82 for expressing ACMSD.


SEQUENCE LISTING (16 sequences)

SEQ ID NO: 1 - DNA, 3251 bp, synthetic construct
  source 1..3251: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtgctagcaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagtattcctcgaagatctgctagcaggcgcgcggccgccgccaccatgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtgagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttactagtaaagcttgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 2 - DNA, 3190 bp, synthetic construct
  source 1..3190: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtgctagcaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacaatgaaaattgacatccatagtcatattctaccaaaagaatggccagatctaaaaaagaggtttggctacggaggctgggtgcagctccaacaccacagcaagggagaagcaaagttgttgaaagatgggaaagtcttcagagtggtgcgagagaattgctgggatccagaagttcgtattagagaaatggaccaaaaaggagtaacagtgcaagccctttccacagttcctgtcatgtttagctactgggccaaacctgaggacactttaaacctgtgccagcttttaaacaacgaccttgccagcaccgttgtgagctaccccaggaggttcgtgggtctggggacgttgcccatgcaggcccctgagctggcggtcaaggagatggagcgctgtgtgaaagagctgggctttcccggggtccaaattggcacccacgtcaacgagtgggacctgaacgcgcaggagctctttcctgtctatgcggcagccgaaaggctgaagtgttccctgttcgtgcatccctgggacatgcagatggatggacgaatggccaaatactggctcccttggcttgtaggaatgccagcagagaccaccatagccatttgctccatgatcatgggtggagtatttgagaagtttcccaaactgaaagtgtgtttcgcacatggtggtggtgccttccccttcacagtgggaagaatctcccatggattcagcatgcgcccagatctgtgtgcccaggacaaccccatgaacccgaagaaataccttggttccttttacacagatgctttggttcatgatcctctgtccctcaagctgttaacagatgtcataggaaaggataaagtcattttgggaaccgattacccctttccactaggtgagctggaacctgggaaactaatagagtccatggaagaatttgatgaagaaacaaagaataaactcaaagccggcaatgccctggcatttttgggtcttgagagaaaacaatttgaatgagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 3 - DNA, 132 bp, synthetic construct
  source 1..132: mol_type = other DNA
aacccctagtgatggagttggccactccctctctgcgcgctcgctcgctcactgaggccgcccgggcaaagcccgggcgtcgggcgacctttggtcgcccggcctcagtgagcgagcgagcgcgcagagagg

SEQ ID NO: 4 - DNA, 1011 bp, synthetic construct
  source 1..1011: mol_type = other DNA
atgaaaattgacatccatagtcatattctaccaaaagaatggccagatctaaaaaagaggtttggctacggaggctgggtgcagctccaacaccacagcaagggagaagcaaagttgttgaaagatgggaaagtcttcagagtggtgcgagagaattgctgggatccagaagttcgtattagagaaatggaccaaaaaggagtaacagtgcaagccctttccacagttcctgtcatgtttagctactgggccaaacctgaggacactttaaacctgtgccagcttttaaacaacgaccttgccagcaccgttgtgagctaccccaggaggttcgtgggtctggggacgttgcccatgcaggcccctgagctggcggtcaaggagatggagcgctgtgtgaaagagctgggctttcccggggtccaaattggcacccacgtcaacgagtgggacctgaacgcgcaggagctctttcctgtctatgcggcagccgaaaggctgaagtgttccctgttcgtgcatccctgggacatgcagatggatggacgaatggccaaatactggctcccttggcttgtaggaatgccagcagagaccaccatagccatttgctccatgatcatgggtggagtatttgagaagtttcccaaactgaaagtgtgtttcgcacatggtggtggtgccttccccttcacagtgggaagaatctcccatggattcagcatgcgcccagatctgtgtgcccaggacaaccccatgaacccgaagaaataccttggttccttttacacagatgctttggttcatgatcctctgtccctcaagctgttaacagatgtcataggaaaggataaagtcattttgggaaccgattacccctttccactaggtgagctggaacctgggaaactaatagagtccatggaagaatttgatgaagaaacaaagaataaactcaaagccggcaatgccctggcatttttgggtcttgagagaaaacaatttgaatga

SEQ ID NO: 5 - DNA, 834 bp, Homo sapiens
  source 1..834: mol_type = other DNA
atgggaaagtcttcagagtggtgcgagagaattgctgggatccagaagttcgtattagagaaatggaccaaaaaggccaaacctgaggacactttaaacctgtgccagcttttaaacaacgaccttgccagcaccgttgtgagctaccccaggaggttcgtgggtctggggacgttgcccatgcaggcccctgagctggcggtcaaggagatggagcgctgtgtgaaagagctgggctttcccggggtccaaattggcacccacgtcaacgagtgggacctgaacgcgcaggagctctttcctgtctatgcggcagccgaaaggctgaagtgttccctgttcgtgcatccctgggacatgcagatggatggacgaatggccaaatactggctcccttggcttgtaggaatgccagcagagaccaccatagccatttgctccatgatcatgggtggagtatttgagaagtttcccaaactgaaagtgtgtttcgcacatggtggtggtgccttccccttcacagtgggaagaatctcccatggattcagcatgcgcccagatctgtgtgcccaggacaaccccatgaacccgaagaaataccttggttccttttacacagatgctttggttcatgatcctctgtccctcaagctgttaacagatgtcataggaaaggataaagtcattttgggaaccgattacccctttccactaggtgagctggaacctgggaaactaatagagtccatggaagaatttgatgaagaaacaaagaataaactcaaagccggcaatgccctggcatttttgggtcttgagagaaaacaatttgaa

SEQ ID NO: 6 - protein, 278 aa, Homo sapiens
MGKSSEWCERIAGIQKFVLEKWTKKAKPEDTLNLCQLLNNDLASTVVSYPRRFVGLGTLPMQAPELAVKEMERCVKELGFPGVQIGTHVNEWDLNAQELFPVYAAAERLKCSLFVHPWDMQMDGRMAKYWLPWLVGMPAETTIAICSMIMGGVFEKFPKLKVCFAHGGGAFPFTVGRISHGFSMRPDLCAQDNPMNPKKYLGSFYTDALVHDPLSLKLLTDVIGKDKVILGTDYPFPLGELEPGKLIESMEEFDEETKNKLKAGNALAFLGLERKQFE

SEQ ID NO: 7 - DNA, 669 bp, synthetic construct
  source 1..669: mol_type = other DNA
tagatctgaattcggtgctagcaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 8 - DNA, 2903 bp, synthetic construct
  source 1..2903: mol_type = other DNA
tagatctgaattcggtgctagcaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacaatgaaaattgacatccatagtcatattctaccaaaagaatggccagatctaaaaaagaggtttggctacggaggctgggtgcagctccaacaccacagcaagggagaagcaaagttgttgaaagatgggaaagtcttcagagtggtgcgagagaattgctgggatccagaagttcgtattagagaaatggaccaaaaaggagtaacagtgcaagccctttccacagttcctgtcatgtttagctactgggccaaacctgaggacactttaaacctgtgccagcttttaaacaacgaccttgccagcaccgttgtgagctaccccaggaggttcgtgggtctggggacgttgcccatgcaggcccctgagctggcggtcaaggagatggagcgctgtgtgaaagagctgggctttcccggggtccaaattggcacccacgtcaacgagtgggacctgaacgcgcaggagctctttcctgtctatgcggcagccgaaaggctgaagtgttccctgttcgtgcatccctgggacatgcagatggatggacgaatggccaaatactggctcccttggcttgtaggaatgccagcagagaccaccatagccatttgctccatgatcatgggtggagtatttgagaagtttcccaaactgaaagtgtgtttcgcacatggtggtggtgccttccccttcacagtgggaagaatctcccatggattcagcatgcgcccagatctgtgtgcccaggacaaccccatgaacccgaagaaataccttggttccttttacacagatgctttggttcatgatcctctgtccctcaagctgttaacagatgtcataggaaaggataaagtcattttgggaaccgattacccctttccactaggtgagctggaacctgggaaactaatagagtccatggaagaatttgatgaagaaacaaagaataaactcaaagccggcaatgccctggcatttttgggtcttgagagaaaacaatttgaatgagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttt

SEQ ID NO: 9 - DNA, 386 bp, synthetic construct
  source 1..386: mol_type = other DNA
tagatctgaattcggtgctagcaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 10 - DNA, 283 bp, synthetic construct
  source 1..283: mol_type = other DNA
catggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 11 - DNA, 94 bp, synthetic construct
  source 1..94: mol_type = other DNA
ggagtcgctgcgacgctgccttcgccccgtgccccgctccgccgccgcctcgcgccgcccgccccggctctgactgaccgcgttactcccacag

SEQ ID NO: 12 - DNA, 924 bp, synthetic construct
  source 1..924: mol_type = other DNA
gtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctaca

SEQ ID NO: 13 - DNA, 205 bp, synthetic construct
  source 1..205: mol_type = other DNA
gcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttt

SEQ ID NO: 14 - DNA, 810 bp, synthetic construct
  source 1..810: mol_type = other DNA
gctcctgggcaacgtgctggttattgtgctgtctcatcattttggcaaagtattcctcgaagatctgctagcaggcgcgcggccgccgccaccatgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtga

SEQ ID NO: 15 - DNA, 262 bp, synthetic construct
  source 1..262: mol_type = other DNA
ttactagtaaagcttgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctagg

SEQ ID NO: 16 - DNA, 105 bp, synthetic construct
  source 1..105: mol_type = other DNA
aattcatatttgcatgtcgctatgtgttctgggaaatcaccataaacgtgaaatgtctttggatttgggaatcttataagttctgtatgagaccactcggatccg